SEQUENCE LISTING (37 sequences)

SEQ ID NO: 1 - DNA, 516 bp, Neisseria meningitidis
tatgccgccgaacgcattacccacaattatccggaaccaaccggtgcagacaaagacaaaataagcacagtaagcgattatttcagaaacatccgtgcgcattccatccaccctcgggtgtcggtcggctacgacttcggcgactggagaatagcggcagattatgccagttacagaaaatggaaagaaagtaattcttctactaatgcagaaaatagagataatgcaaaaaactacgtaaagattgaaacaaaacatcaaggaaacggcagcttccacgccgcttcttctctcggcttatccgccatttacgatttcaaactcaacgataaattcaaaccctatatcggcgcgcgcgtcgcctacggacacgttaaacatcaggttcattcagtggaaacaaaaaccacgactgttacctctaaaccgacggcaacctctccacagggaggccctattatacaaactgatcccagcaaacctccctatcacgaaagccacagcatcagcagcttg

SEQ ID NO: 2 - DNA, 525 bp, Neisseria meningitidis
tatgccgccgaacgcattacccacgattatccgaaagcaaccggtgcaaacaacacaagcacagtaagcgactatttcagaaacatccgtgcgcattccatccacccccgggtgtcggtcggctacgatttcggcggctggaggatagcggcagattatgccagttacagaaaatggaacaacaataaatattccgtaaacacaaaagagttggaaagaaacgatagcaatggcaactggaaagaactgaagacggaaaatcagggaaacggcagcttccacgccgcttcttctctcggtttatccgccatttacgatttcaaactcaacgataaattcgataaattcaaaccctatatcggtgcgcgcgtcgcctacggacacgttaaacatcaggttcattcggtggaaacagaaaccacgactgttttcagtaaaccacaaggcactacagtgccaagcaagatcgtatcaaaagatcccagcaaacctgcctatcacgaaagcaacagcatcagcagcttg

SEQ ID NO: 3 - DNA, 531 bp, Neisseria meningitidis
tatgccgccgaacgcattacccacgattatccgaaaccaaccgatgcaaacaacacaagcacagtaagcgattatttcagaaacatccgtgcgcattccatccaccctcgtgtgtcggtcggctacgacttcggcggctggagaatagcggcagattatgccggttacagaaaatggaacgacaataaatattccgtcaacacaaaagaggtgctaagaaacaatagcactggcactgaaaactggcaagaactgaagacggaaaatcaggaaaacggcagcttccacgccgcttcttctctcggcttatccgccatttacgatttcaaactcaacgataaattcgataaattcaaaccctatatcggcgcgcgcgtcgcctacggacacgttaaacatcaggttcattcggtggaaacagaaaccacgactgttttcagtaaaccaaaaggcactacagtgccaggcattatctcagaaaaaccgattagcaaacctccctatcacgaaagcaacagcatcagcagcttg

SEQ ID NO: 4 - DNA, 510 bp, Neisseria meningitidis
tatgccgccgaacgcattacccacgattatccgcaagcaaccggtgcaaacaacacaagcacggtaagcgattatttcagaaacatccgtgcgcattccatccacccccgggtgtcggtcggctatgatttcggcggctggagaatagcggcagattatgccagttacagaaaatggaaagaaagtaattcttctactaaaaaagttactgaagagataaacaacaactacaaagaaacccaaacaaaacatcagggaaacggcagcttccacgccgtttcttctctcggcttgtccgccatttacgatttcaaactcaacgataaattcaaaccctatatcggcgcgcgcgtcgcctacggacacgttaaacatcaagttcattcggtggaaacaaaaaccacgactattacctctaaaccaaagaacggctctccacagggaggccctattatacaaactgatcccagcaaacctccctatcacgaaagccacagcatcagcagcgtg

SEQ ID NO: 5 - DNA, 525 bp, Neisseria meningitidis
tacgccgccgaacgcattacccacaattatccggaaccaaccggtgcagacaaagacaaaataagcacagtaagcgattatttcagaaacatccgtgcgcattccatccacccccgggtgtcggtcggctacgactttggcggctggagaatagcggcagattatgcccgttacagaaaatggaacaacaataaatattccgtcaacacaaaaaatgtgcaaaaaaacgacaatggcaacaggcaagacctgaagacggaaaatcaggaaaacggtacattccacgccgtttcttctctcggcttatccgccatttacgatttcaaactcaacgataaattcgataaattcaaaccctatatcggtgtgcgcgtcgcctacggacacgtcagacacagcatcgattcgaccaaaaaaacaacaaatgttgttaccgtcgccggtgctgctaacacagcacctacgatttattatgcaccagagacgcaaaacgcctatcacgaaagccacagcatccgccgcttg

SEQ ID NO: 6 - DNA, 510 bp, Neisseria meningitidis
tatgccgccgaacgtattacccacgattatccgaaagcaaccggtgcaaacaacacaagcacagtaagcgattatttcagaaacatccgtgcgcattccatccacccccgggtgtcggtcggctacgatttcggcgactggagaatagcggcagattatgccagttacagaaaatggaacaacaataaatattccgtcaacacaaaagagttggaaaacaagcataacaataagaaagacctgaagacggaaaatcaggaaaacggtacattccacgccgcttcttctctcggcttatccgccatttacgatttcaaactcaacgataaattcaaaccctatatcggtgcgcgcgtcgcctacggacacgtcagacacagcatcgattcgaccaaaaaaacaacagaggttgttacctccacccatggtggtgctgacacaaaacctacgatttataatggggaaagtacgcaaaacgcctatcacgaaagccacagcatccgccgcttg

SEQ ID NO: 7 - DNA, 513 bp, Neisseria meningitidis
tatgccgccgaacgtattacccacgattatccgaaaccaaccggtacagacaaagacaaaataagcacagtaagcgattatttcagaaacatccgtacgcattccatccacccccgggtgtcggtcggctacgatttcggcgactggaggatagcggcagattatgccagttacagaaagtggaacaacaataaatattccgtcaacataaaaaaggagagggaaacccaggacaatagggaagaactgaagacggaaaatcaggaaaacggtacattccacgcagtctcatcgctcggcttatccgccatttacgatttcaaactcaacgataaattcgataaattcaaaccctatatcggtgcgcgcgtcgcctacggacacgttaaacatcaggttcattcggtggaaagcaaaaccacgattgttacctctaaaccaagtggaggccctgtcatacaaggtccgacccacaaacctgcctatcacgaaagccacagcatcagcagcttg

SEQ ID NO: 8 - DNA, 528 bp, Neisseria meningitidis
tatgccgccgaacgcattacccacgattatccgaaagcaaccggtgcaaacaacacaagcacagtaagcgattatttcagaaacatccgtgcgcattccatccacccccgggtgtcggtcggctacgatttcggcggctggaggatagcggcagattatgccagttacagaaaatggaacaacaataaatattccgtaaacacaaaagagttgcaaaaaaacaatagcagtggcatctggcaagaactgaagacggaaaatcaggaaaacggtacattccacgccgcttcttctctcggcttatccgccatttacgatttcaaactcaacgataaattcgataaattcaaaccctatatcggtgcgcgcgtcgcctacggacacgttaaacatcaggttcattcggtgagaaaagaaaccacgactactttcagtccaccagcgcaaggcgctacagtgccaggcaaaatcgtacaaggtccgaccaacaaacctgcctatcacgaaagcaacagtatcagcagctta

SEQ ID NO: 9 - DNA, 507 bp, Neisseria meningitidis
tatgccgccgaacgtattacccacgattatccgaaagcaaccggtgcaaacaacacaagcacagtaagcgattatttcagaaacatccgtgcgcattccatccacccccgggtgtcggtcggctacgatttcggcgactggagaatagcggcagattatgccagttacagaaaatggaacaacaataaatattccgtaaacacaaaagagttggaaaacaagaataacaataagagagacctgaagacggaaaatcaggaaaacggcagcttccacgccgcttcttctctcggcttatccgccatttacgatttcaaactcaacgataaattcaaaccctatatcggtgcgcgcgtcgcctacggacacgtcagacacagcatcgattcgaccaaaaaaacaacagaggttcttacctcctcccatgctcctggcacagcacctacgatttataatcagggaagtacgcaaaacgcctatcacgaaagccacagcatccgccgctta

SEQ ID NO: 10 - DNA, 528 bp, Neisseria meningitidis
tatgccgccgaacgtattacccacgattatccgaaagcaaccggtacagacaaagacaaaataagcacagtaagcgattatttcagaaacatccgtgcgcattccatccacccccgagtgtcagtcggctacgatttcggcggctggaggatagcggcagattatgccagttacagaaaatggagcaacaataaatattccgtcaacacaaaagtgttgaaagaaaaccagggcaacaggataaaactgaagacggaaaatcagggaaacggtacgttccacgcctcttcttctctcggcttatccgccatttacgatttcaaactcaacgataaattcgataaattcaaaccctatatcggtgcgcgcgtcgcctacggacacgttaaacatcaggttcattcggtggaaaccaaaaccacgatttataccactgcaccaacgggagacgctacagtgggaggcactatcccagagagaccgagtagcaaacctgcctatcacgaaagcaacagcatcagcagcttg

SEQ ID NO: 11 - DNA, 534 bp, Neisseria meningitidis
tatgccgccgaacgcattacccacgattatccgaaagcaaccggtgcaaacaacacaagcacagtaagcgattatttcagaaacatccgtgcgcattccatccacccccgggtgtcggtcggctacgatttcggcgactggaggatagcggcagattatgccagttacagaaaatggaacaacaataaatattccgtaaacacaaaagaggtgggaagaaacaataacaataacattgacatctggaaagaactgaagacggaaaatcaggaaaacggcagcttccacgccgcttcttctctcggcttatccgccatttacgatttcaaactcaacgataaattcgataaattcaaaccctatatcggtgcgcgcgtcgcctacggacacgttaaacatcaggttcattcggtgagaaaagaaaccacgactgttttcagtaaaccaagtggtagcgttacaaagccaggcgagatcgtagaaactccgaccagcaaacctgcctatcacgaaagccacagtatcagcagcttg

SEQ ID NO: 12 - DNA, 519 bp, Neisseria meningitidis
tacgccgccgaacgcattacccacgattatccgcaagcaaccggtgcaaacaacacaagcacagtaagcgattatttcagaaacatccgtgcgcattccatccacccccgggtgtcggtcggctacgactttggcggctggagaatagcggcagattatgcccgttacagaaaatggaacaacaataaatattccgtcaacacaaaaaatgtgcaaaaaaacgacaatggcaacaggcaagacctgaagacggaaaatcaggaaaacggtacattccacgccgtttcttctctcggcttatccgccatttacgatttcaaactcaacgataaattcgataaattcaaaccctatatcggtgtgcgcgtcgcctacggacacgtcagacacagcatcgattcgaccaaaaaaacaacaaatgttgttaccgtcgccggtgctgctaacacagcacctacgatttattatgcaccagagacgcaaaacgcctatcacgaaagccacagcatccgccgcttg

SEQ ID NO: 13 - DNA, 510 bp, Neisseria meningitidis
tatgccgccgaacgcattacccacgattatccgcaagcaaccggtgcaaacaacacaagcacggtaagcgattatttcagaaacatccgtgcgcattccatccacccccgggtgtcggtcggctatgatttcggcggctggagaatagcggcagattatgccagttacagaaaatggaaagaaagtaattcttctactaaaaaagttactgaagagataaacaacaactacaaagaaacccaaacaaaacatcagggaaacggcagcttccacgcctcttcttctctcggcttatccgccatttacgatttcaaactcaacgataaattcaaaccctatatcggcgcgcgcgtcgcctacggacacgttaaacatcaagttcattcggtggaaaccaaaaccacgattgttacctctaaaccaacgcaaggcgctgcacagggaggtcctattatacaaactgatcccagcaaacctccctatcacgaaagccacagcatcagcagcgtg

SEQ ID NO: 14 - DNA, 516 bp, Neisseria meningitidis
tacgccgccgaacgcattacccacgattatccgcaagcaaccggtgcaaacaacacaagcacagtaagcgattatttcagaaacatccgtacgcattccatccaccctcgggtgtcggtcggctacgatttcggcgactggaggatagcggcagattatgccagttacagaaaatggaacgacaataaatattccgtcaacacaaaaaatgtgcaggtgaataaaagcaatggcaacaggcaagacctgaagacggaaaatcaggaaaacggtacattccacgccgtctcctcgctcggcttgtccgccgtttacgatttcaataccggttcccgcttcaaaccctatgcaggcgtgcgcgtcgcctacggacatgttagacacagcatcgattcgaccaaaaaaacaacaaatgttcttaccgtccccaccaatattcctggcggaacacctacgatttataatcagggaagtacgcaagacgcctatcacgaaagccacagcatccgccgcttg

SEQ ID NO: 15 - DNA, 525 bp, Neisseria meningitidis
tacgccgccgaacgcattacccacgattatccgcaagcaaccggtgcaaacaacacaagcacagtaagcgattatttcagaaacatccgtgcgcattccatccacccccgggtgtcggtcggctatgatttcggcggctggaggatagcggcagattatgccagttacagaaaatggaacaacaataaatattccgtcaacacaaaagaggtggaaagaaacaataccagtggcaactggaaagaactgaagacggaaaatcaggaaaacggtacattccacgcctcttcttctctcggcttatccgccatttacgatttcaaactcaacgataaattcgataaattcaaaccctatatcggtgtgcgcgtcgcctacggacacgttaaacatcaggttcattcagtgagaaaagaaaccacgactgctttccttaaaccatcgaaaggtgctacagagccaggcaagatcccaagtttggttaccaaacctgcctatcacgaaagccacagcaccagcagcttg

SEQ ID NO: 16 - DNA, 537 bp, Neisseria meningitidis
tatgccgccgaacgcattacccacaattatccggaaccaaccggtgcagacaaagacaaaataagcacagtaagcgattatttcagaaacatccgtgcgcattccatccaccctcgtgtgtcggtcggctacgatttcggcgactggaggatagcggcagattatgccagttacagaaaatggaacaacaataaatattccgtaaacacaaaagaggtgcaaagaaacaatagcactggcactgaaaactggcaagaactgaagacggaaaatcaggaaaacggcagcttccacgccgcttcttctctcggcttatccgccatttacgatttcaaactcaacgataaattcgataaattcaaaccctatatcggtgcgcgcgtcgcctacggacacgttaaacatcaggttcattcagtgagaaaagaaaccacgactgttttcagtaaaccaagaggcattacagagccaggcaaggtcgtaaaaggtccgacctccaaacctccctatcacgaaagccacagcaccagcagcttg

SEQ ID NO: 17 - DNA, 528 bp, Neisseria meningitidis
tatgccgccgaacgcattacccacgattatccgcaagcaaccggtgcaaacaacacaagcacagtaagcgattatttcagaaacatccgtgcgcattccatccacccccgggtgtcggtcggctacgatttcggcggctggaggatagcggcagattatgccagttacagaaaatggagcaacaataaatattccgtaaacacaaaagaggtgcaaagaaacaataacgctggcaactggcaagaactgaagacggaaaatcaggaaaacggcagcttccacgccgtctcttctctcggcttatccgccatttacgatttcaaactcaacgataaattcgataaattcaaaccctatatcggtgcgcgcgtcgcctacggacacgttaaacatcaggttcattcggtgagaaaagaaaccacgactactttcagtaaagcaggggcaggtgctacagagccaggcaaaattatatcaggtccgatctccaaacctgcctatcacgaaagccacagtatcagcagcttg

SEQ ID NO: 18 - DNA, 522 bp, Neisseria meningitidis
tacgccgccgaacgcattacccacgattatccgaaagcaaccggtgcaaacaacacaagcacagtaagcgattatttcagaaacatccgtgcgcattccatccacccccgggtgtcggtcggctacgatttcggcggctggaggatagcggcagattatgccagttacagaaaatggaacaacaataaatattccgtcaacacaaaagtgttgcaagaaaaaaatggcaacaggaaagaactgaagacggaaaatcaggaaaacggtacattccacgccgcttcttctctcggcttatccgccatttacgatttcaaactcaacgataaattcgataaattcaaaccctatatcggcgcgcgcgtcgcctacggacacgttaaacatcaggttcattcagtgaaaaaagaaaccacgactgttttcactgcaccaacgcaaggtgctcaaaagggaggcgttatcccagaagctacgattagcaaacctgcctatcacgaaagccacagcatcagcagcttg

SEQ ID NO: 19 - DNA, 528 bp, Neisseria meningitidis
tacgccgccgaacgcattacccacgattatccgcaagcaaccggtgcaaacaacacaagcacagtaagcgattatttcagaaacatccgtgcgcattccatccacccccgggtgtcggtcggctatgatttcggcggctggaggatagcggcagattatgccagttacagaaaatggaacaacaataaatattccgtaaacacaaaagagttggaaagaaacaataccagtggcaactggaaagaactgaagacggaaaatcaggaaaacggtacattccacgcctcttcttctctcggcttatccgccatttacgatttcaaactcaacgataaattcgataaattcaaaccctatatcggtgcgcgcgtcgcctacggacacgttaaacatcaagttcattcggtggaaaaagaaaccacgactactttcagtaaagcagggctaggtgctacagagccaggcaaaattatatcaggtccgacccccaaacctccctatcacgaaagcaacagcatcagcagcttg

SEQ ID NO: 20 - DNA, 501 bp, Neisseria meningitidis
tatgccgccgaacgtattacccacgattatccgaaaccaaccggtacagacaaagacaaaataagcacagtaagcgattatttcagaaacatccgtacgcattccatccacccccgggtgtcggtcggctatgatttcggcggctggaggatagcggcagattatgccagttacagaaaatggaacaacaataaatattccgtcaacacaaaaaaggtgagagaaaagaatggcaacagggaagacctgaagacggaaaatcaggaaaacggtacgttccacgccgtctcctcgctcggcttatccgccatttacgatttcaaactcaacgataaattcaaaccctatatcggcgcgcgcgtcgcctacggacacgttaaacatcaggttcattcagtggaaagcaaaaccacgactgttacctctaaaggtggaggccctatcccacaaggtccgacccccaaacctccctatcacgaaagccacagcaccagcagcttg

SEQ ID NO: 21 - DNA, 528 bp, Neisseria meningitidis
tatgccgccgaacgtattacccacgattatccgaaagcatccggtgcaaacaacacaagcacagtaagcgattatttcagaaacatccgtgcgcattccatccacccccgggtgtcggtcggctacgacttcggcgactggaggatagcggcagattatgccagttacagaaaatggaacaacaataaatattccgtcaacacaaaagaggtgagaagaaacaataacgctggcaactggcgagaactgaagacggaaaatcaggaaaacggcagcttccacgcctcttcttctctcggcttatccgccatttacgatttcaaactcaacgataaattcgataaattcaaaccctatatcggtgtgcgcgtcgcctacggacacgttaaacatcaggttcattcagtgaaaaaagaaaccacgactactttccttgcaccaacgggagacgctaaagtgccaggcaagatcgtagaaggtccgttcagcaaacctgcctatcacgaaagccacagcatcagcagcttg

SEQ ID NO: 22 - DNA, 516 bp, Neisseria meningitidis
tatgccgccgaacgcattacccacaattatccggaaccaaccggtgcagacaaagacaaaataagcacagtaagcgattatttcagaaacatccgtgcgcattccatccacccccgggtgtcggtcggctacgatttcggcggctggaggatagcggcagattatgccagttacagaaaatggaaagaaagtaatttttctactaaaaaagttactgaagagataaaagacaactacaaagaaaccaaaacagaacatcaaggaaacggcagcttccacgccacttcttctctcggcttatccgccatttacgatttcaaactcaacgataaattcaaaccctatatcggtgcgcgcgtcgcctacgggcacgttaaacatcaggttcattcggtggaaacaaaaaccacgactgttacctctaaaccaaaggggggcactccagctggaggccctgttataaaaactgatcccagcaaacctccctatcacgaaagccacagcatcagcagcttg

SEQ ID NO: 23 - DNA, 522 bp, Neisseria meningitidis
tacgccgccgaacgcattacccacgattatccgaaagcaaccggtgcaaacaacacaagcacagtaagcgattatttcagaaacatccgtgcgcattccatccacccccgggtgtcggtcggctacgatttcggcggctggaggatagcggcagattatgccagttacagaaaatggaacaacaataaatattccgtcaacacaaaagaggtgctaagaaacaataatggcaacaggaaagaactgaagacggaaaatcaggaaaacggtacattccacgcagtctcatcgctcggcttatccgccatttacgatttcaaactcaacgataaattcgataaattcaaaccctatatcggtgcgcgcgtcgcctacggacacgttaaacatcaggttcattcggtggaaacagaaaccacgactgttttcagtaaaccacaaggcactacagtgccaagcaagatcgtatcaaaagatcccagcaaacctgcctatcacgaaagccacagcatcagcagcttg

SEQ ID NO: 24 - DNA, 525 bp, Neisseria meningitidis
tacgccgccgaacgcattacccacgattatccgcaagcaaccggtgcaaacaacacaagcacagtaagcgattatttcagaaacatccgtgcgcattccatccacccccgggtgtcggtcggctacgatttcggcggctggaggatagcggcagattatgccagttacagaaaatggaacaacaataaatattccgtaaacacaaaagagttggaaagaaacgatagcaatggcaactggaaagaactgaagacggaaaatcagggaaacggcagcttccacgccgcttcttctctcggtttatccgccatttacgatttcaaactcaacgataaattcgataaattcaaaccctatatcggtgcgcgcgtcgcctacggacacgttaaacatcaggttcattcggtggaaacagaaaccacgactgttttcagtaaaccacaaggcactacagtgccaagcaagatcgtatcaaaagatcccagcaaacctgcctatcacgaaagcaacagcatcagcagcttg

SEQ ID NO: 25 - DNA, 522 bp, Neisseria meningitidis
tatgccgccgaacgcattacccacgattatccgaaaccaaccggtgcaaacaacacaagcacagtaagcgattatttcagaaacatccgtgcgcattccatccacccccgagtgtcagtcggctacgatttcggcgactggaggatagcggcagattatgccagttacagaaaatggaacaacaataaatattccgtcaacacaaaagaggtgagaagaaacgggggcaacagtgaaaacctgaagacggaaaatcaggaaaacggcagcttccacgccgtctcttctctcggcttatcagccatttacgatttcaaactcaacgataaattcgataaattcaaaccctatatcggtgcgcgcgtcgcctacggacacgttaaacatcaggttcattcggtggaaacagaaaccaagattattttcactacaccaacgaaaggcgctaaagttggaggcacgatcctagacaaatcgatgagcaaacctccctatcacgaaagcaacagcatcagcagcttg

SEQ ID NO: 26 - DNA, 528 bp, Neisseria meningitidis
tatgccgccgaacgcattacccacgattatccgaaagcaaccggtgcaaacaacacaagcacagtaagcgattatttcagaaacatccgtgcgcattccatccacccccgagtgtcagtcggctacgatttcggcgactggagaatagcggcagattatgccagttatagaaaatggaacaacaataaatattccgtcaacacaaaagagttgcaaagaaacaatagcagtggcatctggcaagaactgaagacggaaaatcaggaaaacggtacattccacgccgtctcatcgctcggcttatccgccatttacgatttcaaactcaacgataaattcgataaattcaaaccctatatcggcgcgcgcgtcgcctacggacacgttaaacatcaggttcattcggtgagaaaagaaaccaagactactttcagtaaaccaagggttggcgctacagagccaggcaagatcgtagaaggtccgacccccaaacctccctatcacgaaagcaacagtatcagcagcttg

SEQ ID NO: 27 - DNA, 531 bp, Neisseria meningitidis
tacgccgccgaacgcattacccacgattatccgcaagcaaccggtgcaaacaacacaagcacagtaagcgattatttcagaaacatccgtgcgcattccatccaccctcgtgtgtcggtcggctacgacttcggcggctggagaatagcggcagattatgccagttacagaaaatggaacgacaataaatattccgtcaacacaaaagaggtgctaagaaacaatagcactggcactgaaaactggcaagaactgaagacggaaaatcaggaaaacggtacattccacgccgcttcttctctcggcttatccgccatttacgatttcaaactcaacgataaattcgataaattcaaaccctatatcggcgcgcgcgtcgcctacggacacgttaaacatcaggttcattcggtggaaacagaaaccacgactgttttcagtaaaccaaaaggcactacagtgccaggcattatctcagaaaaaccgattagcaaacctccctatcacgaaagcaacagcatcagcagcttg

SEQ ID NO: 28 - DNA, 495 bp, Neisseria meningitidis
tacgccgccgaacgcattacccacgattatccgcaagcaaccggtgcaaacaacacaagcacagtaagcgattatttcagaaacatccgtgcgcattccatccacccccgggtgtcggtcggctacgatttcggcgactggaggatagcggcagattatgccagttacagaaaatggaaagaaagtaattattctaaaaaagttactgaatttaaacaccaaaacggcaacaaacaagaagacaaaacagaacatcagggaaacggcagcttccacgccacttcttctctcggcttatccgccatttacgatttcaaactcaacgataaattcaaaccctatatcggcgtgcgcgtcgcctacggacacgttaaacatcaggttcattcagtggaaagcaaaaccacgactgttaccactaacaatggaggccctgtcccacaaggtccgacccccaaacctgcctatcacgaaagccacagcatcagcagcgtg

SEQ ID NO: 29 - DNA, 522 bp, Neisseria meningitidis
tatgccgccgaacgtattacccacgattatccgaaagcaaccggtgcaaacaacacaagcacagtaagcgattatttcagaaacatccgtgcgcattccatccaccctcgtgtgtcggtcggctacgatttcggcggctggaggatagcggcagattatgccagttacagaaaatggaacaacaataaatattccgtcaacacaaaagagttggaaaacaagcataacaataagaaagacctgaagacggaaaatcaggaaaacggtacgttccacgccgcttcttctctcggcttatccgccatttacgatttcaaactcaacgataaattcgataaattcaaaccctatatcggtgtgcgcgtcgcctacggacacgttaaacatcaggttcattcagtggaaagcaaaaccacgattgttacctctaaaccaacgaaaggtgctacacagccaggcaagcttgtatcaggtccgacccccaaacctgcttatcacgaaagcaacagcatcagcagcttg

SEQ ID NO: 30 - DNA, 531 bp, Neisseria meningitidis
tacgccgccgaacgtattacccacgattatccgaaaccaaccggtacagacaaagacaaaataagcacagtaagcgattatttcagaaacatccgtgcgcattccatccacccccgggtgtcggtcggctacgatttcggcggctggaggatagcggcagattatgccagttacagaaaatggaacaacaataaatattccgtcaacacaaaagaggtgctaagaaacaataatggcaacaggaaagaactgaagacggaaaatcaggaaaacggtacattccacgcagtctcatcgctcggcttatccgccatttacgatttcaaactcaacgataaattcgataaattcaaaccctatatcggtgcgcgcgtcgcctacgggcacgttaaacatcaggttcattcggtggaaagcaaaaccacgattgttaccactgcaccaacgcaaggtgctccaaagggaggcactatcctacaaggtccgacccccaaacctccctatcacgaaagcaacagcatcagcagcttg

SEQ ID NO: 31 - DNA, 536 bp, Neisseria meningitidis
tatgccgccgaacgtattacccacgattatccgaaagcaaccggtgcagacaaagacaaaataagcacagtaagcgattatttcagaaacatccgtgcgcattccatccaccctcgtgtgtcggtcggctacgacttcggcggctggagaatagcggcagattatgccagttacagaaaatggaacaacaataaatattccgtcaacacaaaagagttgcaaagaaacaaggagcatggcaactggaaagaactgaagacggaaaatcaggaaaacggcagcttccacgccgcttcttctctcggcttatccgccatttacgatttcaaactcaacgataaattcgataaattcaaaccctatatcggtgtgcgcgtcgcctacggacacgttaaacatcaggttcattcagtgaaaaaagaaaccacgactactttccttgcaccaacgggagacgctaaagtgccaggcaagatcgtagaaggtccgttcagcaaacctgcctatcacgaaagccacagcaccagcagcttggt

SEQ ID NO: 32 - DNA, 522 bp, Neisseria meningitidis
tatgccgccgaacgtattacccacgattatccgaaagcaaccggtgcaaacaacacaagcacagtaagcgattatttcagaaacatccgtgcgcattccatccaccctcgtgtgtcggtcggctacgatttcggcgactggaggatagcggcagattatgccagttacagaaaatggaacaacaataaatattccgtcaacacaaaagaggtgcaaagacatagcaatggcaactggaaagaactgaagacggaaaatcagggaaacggcagcttccacgccgcttcttctctcggtttatccgccatttacgatttcaaactcaacgataaattcgataaattcaaaccctatatcggtgcgcgcgtcgcctacggacacgttaaacatcaagttcattcggtggaaaaagaaaccacgactactttcagtaaagcagggctaggtgctacagagccaggcaagatcccaagtttggttaccaaacctgcctatcacgaaagcaacagcatcagcagcttg

SEQ ID NO: 33 - DNA, 510 bp, Neisseria meningitidis
tatgccgccgaacgtattacccacgattatccgaaagcaaccggtgcaaacaacacaagcacagtaagcgattatttcagaaacatccgtgcgcattccatccacccccgggtgtcggtcggctacgatttcggcggctggaggatagcggcagattatgccagttacagaaaatggaaagaaagtaattcttctactaaaaaagttactgaagatatagcagacaactacaaagaaaccaaaacagaacatcaaggaaacggcagcttccacgccgcttcttctctcggcttatccgccatttacgatttcaaactcaacgataaattcaaaccctatatcggcgcgcgcgtcgcctacggacacgttaaacatcaggttcattcagtggaaacaaaaaccacgactgttacctctaaaccgacggcaacctctccacagggaggccctattatacaaactgatcccagcaaacctccctatcacgaaagccacagcatcagcagcttg

SEQ ID NO: 34 - DNA, 507 bp, Neisseria meningitidis
tacgccgccgaacgcattacccacgattatccgcaagcaaccggtgcaaacaacacaagcacagtaagcgattatttcagaaacatccgtgcgcattccatccaccctcgtgtgtcggtcggctacgatttcggcgactggaggatagcggcagattatgccagttacagaaaatggaacaacaataaatattccgtaaacacaaaagagttggaaaacaagaataacaataagagagacctgaagacggaaaatcaggaaaacggcagcttccacgccgcttcttctctcggcttatccgccatttacgatttcaaactcaacgataaattcaaaccctatatcggtgcgcgcgtcgcctacggacacgtcagacacagcatcgattcgaccaaaaaaacaacagaggttcttacctcctcccatgctcctggcacagcacctacgatttataatcagggaagtacgcaaaacgcctatcacgaaagccacagcatccgccgctta

SEQ ID NO: 35 - DNA, 510 bp, Neisseria meningitidis
tacgccgccgaacgtattacccacgattatccgaaagcaaccggtgcaaacaacacaagcacagtaagcgattatttcagaaacatccgtgcgcattccatccacccccgggtgtcggtcggctacgatttcggcgactggaggatagcggcagattatgccagttacagaaaatggaaagaaagtaattcttctactaatacagaaaatagcgagactcaacagaaccgcataaagattgaaacaaaacatcaaggaaacggcagcttccacgccgcttcttctctcggcttatccgccatttacgatttcaaactcaacgataaattcaaaccctatatcggcgcgcgcgtcgcctacggacacgttaaacatcaggttcattcagtggaaacaaaaaccacgactgttacctctaaaccgacggcaacctctccacagggaggccctattatacaaactgatcccagcaaacctccctatcacgaaagccacagcatcagcagcttg

SEQ ID NO: 36 - DNA, 528 bp, Neisseria meningitidis
tatgccgccgaacgtattacccacgattatccgaaagcaaccggtgcaaacaacacaagcacagtaagcgattatttcagaaacatccgtgcgcattccatccacccccgggtgtcggtcggctacgatttcggcgactggaggatagcggcagattatgccagttacagaaaatggaacaacaataaatattccgtaaacacaaaagagttgctaagaaacaatagcagtggcatctggcaagaactgaagacggaaaatcaggaaaacggtacattccacgccgcttcttctctcggcttatccgccatttacgatttcaaactcaacgataaattcgataaattcaaaccctatatcggtgcgcgcgtcgcctatggacacgttaaacatcaggttcattcagtgagaaaagaaaccaagactactttcagtccaccaaagcaaggcgctacagtgccaggcaagatcgtagaaggtccgttcagcaaacctccctatcacgaaagccacagcatcagcagcttg

SEQ ID NO: 37 - DNA, 507 bp, Neisseria meningitidis
tacgccgccgaacgcattacccacgattatccgcaagcaaccggtgcaaacaacacaagcacagtaagcgattatttcagaaacatccgtgcgcattccatccaccctcgtgtgtcggtcggctacgatttcggcgactggaggatagcggcagattatgccagttacagaaaatggaacaacaataaatattccgtaaacacaaaagagttggaaaacaagaataacaataagagagacctgaagacggaaaatcaggaaaacggcagcttccacgccgcttcttctctcggcttatccgccatttacgatttcaaactcaacgataaattcaaaccctatatcggtgcgcgcgtcgcctacggacacgtcagacacagcatcgattcgaccaaaaaaacaacagaggttcttacctcctcccatgctcctggcacagcacctacgatttataatcagggaagtacgcaaaacgcctatcacgaaagaaacagtatcagcagcttg

(19) United States
(12) Patent Application Publication
(10) Pub. No.: US 2010/0183676 A1
(43) Pub. Date: Jul. 22, 2010

(54) COMPOSITIONS COMPRISING OPA PROTEIN EPITOPES

(75) Inventors: Martin Maiden, Oxford (GB); Ian Feavers, Herts (GB); Andrew Pollard, Oxford (GB)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: ISIS INNOVATION LTD., Oxford (GB)

(21) Appl. No.: 11/722,330

(22) PCT Filed: Dec. 22, 2005
(86) PCT No.: PCT/GB2005/005014

§ 371 Date: Nov. 19, 2009

(30) Foreign Application Priority Data
Dec. 24, 2004 (GB) 0428381.8

(51) Int. Cl. A61K 39/04; C07H 21/00; A61P 31/04; A61P 37/04

(52) U.S. Cl. 424/248.1; 536/23.7

(57) ABSTRACT

The present invention relates to a composition comprising at least one purified Opa HV 1 protein epitope and at least one purified Opa HV2 protein epitope. The epitopes are preferably purified protein epitopes, preferably recombinant epitopes. The composition is preferably a pharmaceutical composition, more preferably a vaccine composition. The invention also relates to methods of immunisation and to specific formulations presented in the tables, and to novel nucleic acids encoding Opa alleles.

### FIELD OF THE INVENTION

The invention relates to pharmaceutical and vaccine compositions for protection against N. meningitidis, especially protection against N. meningitidis serogroup B. In particular the invention relates to meningococcal hyperinvasive lineage specific recombinant Opa protein vaccines.

### BACKGROUND TO THE INVENTION

Neisseria meningitidis, a common commensal inhabitant of the human nasopharynx, is a major cause of bacterial meningitis and septicaemia worldwide. Acapsulate meningococci are essentially avirulent and only five of the thirteen chemically and immunologically distinct meningococcal capsular polysaccharides, which define meningococcal serogroup, are frequently associated with invasive disease. Although protein-polysaccharide conjugate vaccines offer the possibility of protection against meningococcal disease caused by serogroups A, C, Y and W135, this approach has not been successful for serogroup B meningococci. Furthermore, comprehensive prevention does not appear possible with polysaccharide-based vaccines alone. Problems arise from the fact that the serogroup B polysaccharide structure is poorly immunogenic. Further problems arise due to its similarity to sialylated glycopeptides on human cells.

Prior art vaccines have often made use of purification of so called “blebs” which represent vesicles shed from the cell surface of the particular organism of interest. However, such a crude product carries many problems. For example, there is wide variation in the composition of these blebs. There is no reliable way of controlling which proteins are included or excluded from these blebs. These blebs may or may not include polysaccharide-coating elements of the organism of interest. The proportions of the various components of the blebs in relation to one another cannot be reliably determined. The composition of these blebs cannot be easily determined or controlled.

Many attempts have been made to develop vaccines based on the sub-capsular antigens, especially the outer membrane proteins (OMPs). Meningococcal OMPs are highly diverse and, although OMP-containing outer membrane vesicle (OMV) vaccines have been effective against the particular epidemic strain from which they were made, levels of potentially cross-protective immune responses to heterologous strains have been disappointing.

OMV vaccines containing outer membrane proteins (OMPs) have been used in the control of epidemics caused by single strains in Norway and Cuba. Due to the high antigenic diversity of many OMPs among different strains, immune responses to vaccines of this type are usually limited to the strains used in their manufacture or their close relatives. Consequently, this approach does not provide effective control of endemic serogroup B disease, which is attributed to diverse strains.

Opacity (Opa) proteins are a family of antigenically variable outer-membrane proteins of Neisseria meningitidis. Even among clonally related epidemic meningococcal isolates, there is greater variation of Opa protein expression than can be accounted for by the opa gene repertoire of any individual strain. Hobbs et al. (1994 Mol. Microbial. April;12(2):171-80) studied microevolution of the opa gene family within a clonal population of N. meningitidis. DNA analysis indicated that changes occurred in the opa genes of these bacteria as they spread through the human population over a relatively short period of time. Additional variability in this gene family appears to have been introduced at least in part by horizontal exchange of opa sequences from other meningococcal strains and from Neisseria gonorrhoeae. Thus the Opa proteins have been shown to be extremely variable, and this variability is thought to contribute to immune evasion.

Mandrell and Zollinger (1989 Infect. Immun. vol 57 pp 1590-1598) describe human immune responses to certain OMPs following infection or vaccination with a polysaccharide-protein complex. Responses to serogroup B and C meningococci are reported.

Milagres et al. (1998 Infect. Immun. vol 66 pp 4755-4761) describe bactericidal antibody responses to serogroup B meningococci following immunisation with the Cuban BC (B:4:P1.15) OMP vaccine. This vaccine is an OMV vaccine. The main bactericidal responses detected were against PorA and class 5 (Opa) proteins, although a quarter of the samples studied had bactericidal responses which could not be attributed to reactions against either of these proteins, and almost half of the samples only had PorA-directed responses.

Malorny et al. (1998 J. Bact. vol 180 pp 1323-1330) describe sequence diversity, structure and epitope mapping of Neisserial Opa proteins. The sequence variation in the surface exposed parts of the Opa proteins is so high that these parts of the protein had to be manually gapped and/or excluded from the analysis in order to make meaningful comparisons.

The present invention seeks to overcome problem(s) associated with the prior art.

### SUMMARY OF THE INVENTION

The present invention is based on the surprising finding that antigenically variable antigens can be exploited in vaccinating to provide protection against a range of different strains of the target organism. Previously, antigenically variable antigens were considered to be very poor candidate components for vaccine compositions due to their variability and various other difficulties which have been outlined above. Variable antigens have previously only been considered suitable for strain-specific vaccination. However, as explained in detail herein, certain combinations of said antigens may be exploited to provide an effective pharmaceutical composition, preferably vaccine composition.

In particular, in the field of meningococcal vaccines, the combination of Opa antigens is found to be particularly advantageous.

Furthermore, the invention is based on deeper insights into the representation of different Opa alleles across the extremely broad range of antigenically diverse meningococcal isolates which have been studied. It is surprisingly shown that selection of a relatively small number of variable Opa antigens can provide protection against a broad range of those disease associated isolates.

In a broad aspect the invention relates to a pharmaceutical composition, preferably a vaccine composition, comprising at least one purified Opa protein antigen. This is advantageous because it avoids problems associated with prior art vesicle/capsule containing formulations. It is surprisingly effective even despite the prior art view that the antigenic diversity of Opa is too high to allow for its use in vaccine compositions, especially preferred compositions covering more than one target isolate.

The Opa genes encode proteins with defined variable domains. The maximally variable domains are the two hypervariable (HV) regions. It is surprisingly shown herein that these variable domains may be used in pharmaceutical compositions, preferably in vaccine compositions. Thus in another aspect the invention relates to a pharmaceutical composition, preferably a vaccine composition, comprising at least one purified Opa HV1 protein epitope and at least one purified Opa HV2 protein epitope.

Opa proteins further comprise a semivariable region (SV). Preferably the pharmaceutical composition, preferably vaccine composition, as described above further comprises at least one purified Opa SV protein epitope. This advantageously provides another epitope for generation of immune response to Opa.

Particularly preferred combinations of Opa epitopes are provided herein, especially in the tables. Thus, in another aspect the invention relates to a composition, preferably a pharmaceutical composition, more preferably a vaccine composition as described above wherein said epitopes are selected from the epitopes defined in Table A and Tables 1-9. The tables discuss compositions designed or optimised for particular isolates or groups of isolates. Thus, preferably said epitopes are selected from the epitopes defined in a single table selected from Table A and Tables 1-9. Maximal coverage may be advantageously obtained by optimising epitope representation in view of the strain(s) being targeted. Thus, preferably said composition comprises each of the epitopes defined in said single table.

The compositions of the invention are preferably immunogenic. Thus in one embodiment, the invention relates to use of a composition according to the present invention to induce an immune response in a subject. In another embodiment the invention relates to a method of immunising a subject or a method of inducing an immune response in a subject comprising administering to said subject an effective amount of a composition according to the present invention.

Opa alleles are presented in the sequence listing. The Opa alleles comprise particular HV (and SV) coding sequences. Therefore the alleles define HV (and SV) epitopes. Thus in another aspect the invention relates to a pharmaceutical composition, preferably a vaccine composition, as described above wherein the composition comprises epitopes encoded by Opa alleles presented in the sequence listing.

The tables discuss compositions designed or optimised for particular isolates or groups of isolates. Thus, in another aspect the invention relates to a pharmaceutical composition, preferably a vaccine composition as described above wherein the composition comprises epitopes encoded by the Opa alleles defined in a single table selected from Table A and Tables 1-9. Maximal coverage may be advantageously obtained by optimising epitope representation in view of the strain(s) being targeted. Thus, preferably said composition comprises each of the epitopes defined in said single table.

It may be advantageous to include further non-Opa elements in the compositions of the present invention. One class of possible further components is further antigens to advantageously enhance the immune response generated and/or to provide further ‘hits’ against the target strain. Thus in another aspect the invention relates to a pharmaceutical composition, preferably a vaccine composition as described above further comprising one or more components selected from the group consisting of transferrin binding proteins, PorB and NspA. Preferably said further component is selected from the group consisting of transferrin binding protein and PorB. Preferably said further component is PorB.

In another aspect the invention relates to a method of immunising a subject against Neisseria meningitidis infection comprising administering to said subject an effective amount of a pharmaceutical composition, preferably vaccine composition as described above. Preferably the subject is a child.

In another aspect the invention relates to a method of inducing an immune response against Neisseria meningitidis in a subject comprising administering to said subject an effective amount of a pharmaceutical composition, preferably vaccine composition as described above.

The specific combinations of Opas exemplified are particularly advantageous and therefore in another aspect the invention relates to a composition comprising a combination of Opa epitopes as set out in a single table selected from Table A and Tables 1-9. In another aspect the invention relates to a composition comprising a combination of Opa epitopes as described herein. Preferably the composition is a pharmaceutical composition. Preferably the composition is a vaccine composition. The composition may be an outer membrane vesicle vaccine. Preferably the composition is of purified proteins and does not comprise significant vesicle or membrane or capsule components.

In another aspect the invention relates to a composition such as a pharmaceutical composition, preferably a vaccine composition, as described above comprising at least one purified protein selected from the group consisting of OpaA, OpaB, OpaD and OpaJ. Preferably the composition comprises at least two purified proteins selected from the group consisting of OpaA, OpaB, OpaD and OpaJ. Preferably the composition comprises at least three purified proteins selected from the group consisting of OpaA, OpaB, OpaD and OpaJ. Preferably the composition comprises purified OpaA, OpaB, OpaD and OpaJ protein. This has the advantageous effect of maximising the number of hits provided by the composition against any one strain since most strains express all four Opa genes and the great majority express at least three of the Opa genes.

In another aspect the invention relates to an isolated nucleic acid comprising nucleotide sequence encoding a N. meningitidis Opa protein, said nucleotide sequence being selected from the Opa allele sequences presented in the sequence listing.

### Opa

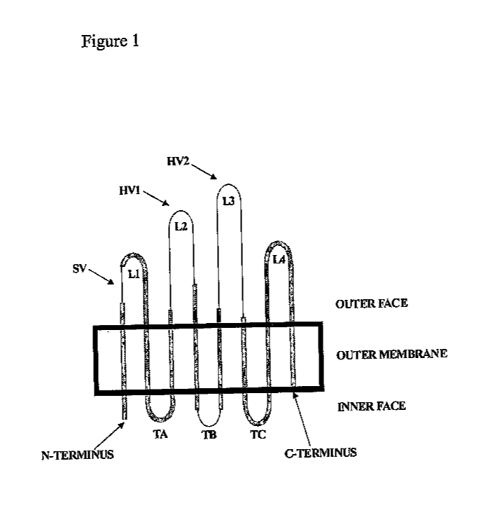

The opacity associated adhesin (Opa) proteins of Neisseria meningitidis are naturally located in the meningococcal outer membrane, are immunogenic and can elicit stronger T-cell responses than some other meningococcal vaccine candidates and components including the PorA protein. FIG. 1 shows predicted two-dimensional structure of Opa proteins showing the predicted 8-stranded β-barrel topology, linked by four putatively surface exposed loop regions and three periplasmic turns. Variable stretches, indicated by a continuous line, are located in three of the four loop regions. The semi variable region is located in loop 1, the hypervariable regions 1 and 2 in loops 2 and 3 respectively.

Opa proteins have a role in entry to and localisation within a host subject. Thus Opa must be ‘on’ to cause disease. By vaccinating/immunising according to the present invention, not only are bactericidal responses promoted, but disease progression and/or invasion/adhesion is also targeted. In this scenario, it will be appreciated that the immune responses generated contribute to produce an effective block to disease progression by targeting that phase of Opa expression through which the pathogen must pass in order to cause disease.

Furthermore, by targeting the molecular basis of adherence/entry in the nasopharynx, herd immunity can be promoted, spread prevented and colonisation reduced, all of which are benefits not found in the prior art.

The function of these Opa proteins in meningococcal adherence provides the advantage that a vaccine directed against Opa may induce both anti-adherence antibodies in the nasopharynx and bactericidal immunity in the blood.

Like most of the surface proteins of N. meningitidis, the Opa proteins are antigenically highly variable. However, we have established that this variability is highly structured such that individual hyperinvasive lineages of meningococci appear to have a limited repertoire of Opa proteins. The proteins are highly conserved in these lineages, which are responsible for most meningococcal disease, during global spread spanning periods of up to three decades. These facts disclosed here for the first time have allowed the invention of a pharmaceutical composition, preferably a vaccine composition, containing the limited number of Opa proteins found only in the hyperinvasive lineages in order to provide protection against the disease-causing bacteria.

Thus the invention relates to use of this limited set of Opa proteins for inclusion in vaccines against hyperinvasive clonal lineages of meningococci. This provides a novel approach that targets only the hyperinvasive meningococci without removing the beneficial effects of nasopharyngeal carriage of non-invasive meningococci that may be involved in boosting immunity to these organisms. Recombinant vaccines containing the Opa proteins specifically targeted at each of the 4 hyperinvasive lineages associated with the serogroup 13 capsule are preferred.

In another aspect the invention relates to a meningococcal hyperinvasive lineage specific recombinant Opa protein vaccine.

Opa proteins are expressed on the surface of meningococci and mediate intimate attachment to the human nasopharynx via interactions with CEACAM proteins expressed on the mucosal epithelia. Thus, antibody raised against them may allow interference with adhesion or, in conjunction with complement, facilitate bacterial killing though lysis or opsonophagocytosis. We disclose herein that the population diversity of opa genes is highly structured, such that each hyperinvasive meningococcal lineage expresses only a limited repertoire of Opa proteins. This repertoire is highly preserved during periods of global spread spanning up to three decades. Thus the invention relates to lineage-specific combinations of Opa proteins in recombinant vaccine compositions directed against meningococcal hyperinvasive lineages.

Other aspects of the invention include construction of a purified protein composition such as a vaccine consisting of Opa proteins from the hyperinvasive lineages of N. meningitidis; evaluation of the immunogenicity of these proteins in animals with respect to antibody induction; evaluation of the functional characteristics of antibody raised in mice that targets the Opa proteins from hyperinvasive lineages of N. meningitidis using serum bactericidal assay and opsonophagocytosis; use of an Opa-based vaccine to induce protective immunity to N. meningitidis.

Based on the observation that hyperinvasive meningococcal lineages have a structured, limited and defined Opa repertoire, we have utilised Opa proteins to develop hyperinvasive lineage-specific, pharmaceutical compositions, preferably vaccine compositions, preferably comprising purified and/or recombinant Opa proteins. Preferably the Opa proteins are purified and recombinant. These compositions are constructed corresponding to the opa gene repertoire of those hyperinvasive lineages known to have been associated with the serogroup B capsular polysaccharide.

This invention brings together expertise in bacterial population biology, protein biochemistry, immunobiology and vaccinology to constitute a novel approach to meningococcal vaccine development by exploiting data from population genetic and molecular evolutionary studies in the design of a vaccine directed against the meningococcal hyperinvasive lineages.

### Opa Combinations

Four opa gene loci exist in the meningococcal chromosome: opaA, opaB, opaD and opaJ. Each opa gene encodes an Opacity (Opa) protein with three variable regions; the semivariable region (SV) and hypervariable regions 1 and 2 (HV1 and HV2 respectively) which are likely to encode the two immunologically dominant epitopes in each Opa protein.

The HVRC (hypervariable region combination) number is a unique number assigned to an individual combination of HV1+HV2 families, eg. HVRC3 (Example 1—Table 1) is the unique identifying number of the HV1:1 A+HV2:8 combination.

Within each HVRC, there may be multiple combinations of individual HV1+HV2 variants, all belonging to the same family in each region, i.e. HVRC3 may include HV1:1 A-1+HV2: 8-1 or HV1:1 A-2+HV2: 8-2. As the HV1+HV2 combination defines the functionality of the Opa protein, HVRC designations are important in understanding and relating the functional properties of different Opa variants and may be used as a basis upon which to further investigate these properties.

In terms of HV epitopes, each opa locus encodes 2 epitopes, the 4 opa loci in each meningococcal chromosome encode a total of 8 epitopes (except in isolates belonging to the subgroup IV-1 clone of the ST-4 complex, which have only 3 loci: the gene at the opaJ locus is deleted). Individual clonal complexes possess a limited and structured repertoire of Opa proteins, and also therefore, of HV1 and HV2 region epitopes. The composition recipes such as vaccine composition recipes herein are designed to induce a response against the HV region epitopes associated with individual lineages and combinations of lineages. In each case, opa alleles encoding the relevant HV1-HV2 variants are included, but it is not absolutely necessary to use the specific alleles given, as any other allele encoding the same HV1-HV2 variants may alternatively be used. Thus it should always be borne in mind that the HV protein epitopes are the essential elements, and the references to nucleic acid alleles are to be regarded as references to the protein epitopes encoded by them.

SV region variants encoded by the alleles are also included which may advantageously provide a third epitope, or set of epitopes if more than one SV region variant is present, in each composition such as vaccine composition.

Various compositions such as Opa vaccines based on HV1 and HV2 region variants are disclosed. Numerous purified Opa-combination vaccine recipes against individual clonal complexes of Neisseria meningitidis (by HV1-HV2 amino acid variants) are described below, in particular in the examples section.

The number of Opa proteins included in a particular composition will depend on the target strain(s) to be covered. Preferably 20 or fewer Opa proteins are included. Preferably 15 or fewer, preferably 13 or fewer, preferably 10 or fewer, preferably 8 or fewer, preferably 6 or fewer, preferably 5 or even fewer. The lower the number of proteins included, the simpler and cheaper the composition is to produce.

### Form of the Antigens

The HV antigens of the present invention may be supplied in any suitable purified form, preferably purified protein form. Purified means removed from the context of the Neisseria organism from which the protein is derived. In particular purified means separated from cellular components such as Neisseria membranes and/or vesicles. Thus the term ‘purified’ in this context means essentially free from cellular components such as polysaccharide capsule material and/or vesicles. Preferably the antigens are used in the form of essentially homogeneous protein preparations as judged by coomassie stained SDS-PAGE, notwithstanding the fact that the individual protein molecules may be diverse such as by comprising different HV epitopes.

Thus preferably the term purified requires that the preparation consist essentially of isolated polypeptide molecules, preferably consisting only of isolated polypeptide molecules with no significant impurities.

Clearly, a purified preparation of one antigen/epitope mixed with a purified preparation of another antigen/epitope to produce a composition such as a vaccine composition gives rise to a mixture of at least two polypeptide species which mixture itself will not be homogeneous since it will comprise at least two antigen species. Thus, a composition comprising purified X and purified Y will be understood to be ‘purified’ in the sense explained above ie. that it will be essentially free from cellular components such as polysaccharide capsule material and/or vesicles. The mere presence in the overall composition of different individual elements X and Y does not mean that those elements are no longer ‘purified’ by virtue only of having been combined as described.

Preferably the antigens/epitopes recombinant. Preferably the antigens/epitopes are produced by recombinant means. Preferably the antigens/epitopes are produced in the absence of N. meningitidis, such as production by recombinant means in a non-N. meningitidis cell. Preferably the antigens/epitopes are produced via expression in E. coli.

The production of the individual purified antigens/epitopes is within the ability of the person skilled in the art. These can be produced by any suitable means known in the art, for example by recombinant expression, purification and refolding (if necessary) in vitro. This is discussed in more detail below.

Antigens/epitopes may be concatenated ie. physically joined for example by production of multiple antigens by expression as a continuous polypeptide chain. This may be done for simple convenience/optimisation of the production process, or for other reasons such as balancing of the induced immune response. Preferably only antigens inducing similar strength responses are concatenated onto single polypeptides. Preferably concatenation is only performed for antigens occurring within a particular protein. Preferably concatenation is avoided. Preferably individual purified antigens are prepared and stored separately until needed to produce a composition according to the present invention.

The precise amounts of individual antigens in particular compositions such as vaccine compositions will typically be determined by the person working the invention. Preferably equimolar amounts of individual antigens are used. More preferably the amounts used are balanced with regard to the strength of immune response induced against a particular antigen species or epitope. Thus, if an antigen elicits a response at only half the strength of a reference antigen, then twice the molar amount of that antigen should be used. Similarly, if an antigen elicits a response at twice the strength of the reference antigen, then approximately half the molar amount should be used. Preferably the optimal relative proportions and dosage levels are determined by clinicians/clinical studies.

This balancing advantageously helps to avoid immunodominance effects produced by inequalities between the individual antigen components of the compositions. This process can be seen as a simple process of titration towards an end-point of even response against each of the antigens in a given composition, preferably of even protection against each of the antigens in a given vaccine composition. The titration is preferably performed by a serum bactericidal antibody assay or an ELISA.

The antigens present in the compositions of the present invention are defined by reference to the HV regions. The HV domains are the important elements of the compositions. The HV regions are part of larger Opa protein molecules. The HV regions mentioned will ordinarily be supplied as integral parts of those Opa molecules. It is possible that the HV regions could be supplied separately as individual purified proteins or fragments/polypeptides. However, it is preferred that the HV antigens are supplied as integral parts of complete Opa proteins. This has the advantage that the folding and presentation of the HV epitopes is in the natural context of the Opa parent protein and advantageously avoids extra labour which might be involved in the refolding/preparation of HV epitopes individually.

It is possible that the HV epitopes may be supplied for in vivo expression/protein production. In this scenario the epitopes are supplied in the form of nucleic acid capable of supporting their expression. Such nucleic acids may comprise those sequences presented in the sequence listing. However, it is preferred that the epitopes are supplied as purified proteins.

The HV epitopes mentioned herein are encoded by the nucleic acids presented in the sequence listing. The nucleic acids presented herein may be translated to give the amino acid sequence of the Opa proteins which they encode. The HV epitopes are thus derived from the nucleic acid sequences presented.

The HV1 and HV2 epitopes may be provided separately. Preferably they are provided on a single Opa protein.

When provided on a single polypeptide, the HV1 and HV2 epitope combinations required may be provided using naturally occurring Opa alleles or by combination of the HV1 and HV2 epitopes into synthetic Opa alleles, for example by nucleic acid shuffling. Preferably naturally occurring Opa alleles are used. Preferably the HV1 and HV2 epitopes are provided in the pairings disclosed in the tables presented herein.

### Opa Genes and HV Epitopes

As will be apparent from the specification, the focus of the invention is on purified Opa proteins and particular Opa HV epitope combinations in the compositions of the invention. As explained herein, most N. meningitidis strains carry four Opa genes. Naturally the best compositions, such as vaccine compositions, according to the present invention are those which direct an immune response against all four Opa gene products. However, it must be borne in mind that recombination events and horizontal transfer have established a situation where HV epitopes/alleles can be shared across Opa loci. For example, with reference to Table A, it can be seen that the Z4699 isolate of the ST-32 complex has Opa allele 185 at both opaB and opaD loci. This again illustrates the importance of reference to the Opa alleles as the determining feature in specifying the particular HV epitopes in the compositions described herein.

Furthermore, it is insights such as this which have advantageously allowed the number of individual epitopes incorporated into each composition to be systematically minimised. For example, by including Opa185 in a composition, immune responses against HVs 1 and 2 of Opa185 are generated, which results in an immediate four hits against Z4699 (HV1 at opaB and at opaD, and HV2 at opaB and at opaD). This approach is applied throughout the examples section and is a key feature underlying the composition such as vaccine composition designs presented herein.

The Opa alleles discussed herein are presented in the sequence listing.

A target N. meningitidis isolate is a particular strain of N. meningitidis against which it is desired to vaccinate/immunise a subject. Preferably the composition comprises at least one HV epitope found at least one Opa locus of a target N. meningitidis isolate. Preferably the composition comprises at least two HV epitopes found at one or more Opa loci of a target N. meningitidis isolate, preferably at least three HV epitopes, preferably at least four HV epitopes, preferably at least five HV epitopes, preferably at least six HV epitopes, preferably at least seven HV epitopes, preferably eight HV epitopes.

Preferably the epitopes are distributed evenly across Opa loci in the target isolate(s), for example the first four epitopes are preferably each in different Opa loci in the target isolate(s), and epitopes 5-8 are preferably each in different Opa loci in the target isolate(s).

Preferably the composition according to the present invention possesses epitopes present in each of the Opa loci present in the target strain.

Preferably the composition according to the present invention possesses epitopes present in each of the four Opa loci A, B, D and J.

By targeting each of the Opa proteins present in the target strain, the coverage of the composition in terms of ‘hits’ per cell is advantageously increased. Advantages which flow from this multiple hit approach include flexibility to design new formulations/compositions in rapid response to a new epidemic or outbreak.

By preparing the Opa proteins as purified proteins, the compositions can be readily standardised. This is in contrast to prior art OMV vaccines which are hard to standardise in terms of their composition and/or behaviour. Furthermore, purified proteins are inherently simpler and safer since they comprise fewer and better defined components. They are also more robust and easier to prepare and store than prior art vesicle based vaccines.

The same considerations apply to any SV epitopes which may be included in the compostion(s).

The advantageous effect of selecting the epitopes according to the present invention is that the coverage in terms of different proteins on the cell surface of the pathogen is maximised. Furthermore, certain phase variation phenomena have been observed with regard to the Opa loci. Advantageously, by following the guidance herein, the probability of generating an immune response against the target pathogen is maximised in the face of possible phase variation effects altering the Opa repertoire expressed at any given time. The compositions according to the examples have been designed to each possess this special technical feature.

### Opa Production

Opa proteins are produced and refolded as required using any suitable techniques known in the art.

Preferably Opa proteins are prepared according to Prince S M, Feron C, Janssens D, Lobet Y, Achtman M, Kusecek B, Bullough P A and Derrick J P (2001 Acta Crystallogr D Biol Crystallogr. August; 57(Pt 8):1164-6 “Expression, refolding and crystallization of the OpcA invasin from Neisseria meningitidis.”).

Others have also published details of these protocols from single meningococcal isolates and their use in immunological studies de Jonge, M. I., et al., (Conformational analysis of opacity proteins from Neisseria meningitidis. European Journal of Biochemistry, 2002. vol 269(21): p. 5215-23) and de Jonge, M. I., et al., (Functional activity of antibodies against the recombinant OpaJ protein from Neisseria meningitidis. Infect Immun, 2003. vol 71(5): p. 2331-40).

Briefly, the protein is overexpressed in Escherichia coli in an insoluble form and then refolded by rapid dilution from denaturant into detergent solution.

### Optimisation

Clearly, the best composition such as vaccine composition may well be the one having broadest coverage. However, to obtain the broadest coverage may require the inclusion of the greatest number of antigens (ie. Opa epitopes) into the composition. Therefore, preferably a balance is struck between minimising the number of epitopes included in the composition and maximising the coverage of protection which might be afforded by said composition. These are the factors which should govern the choice of epitopes in the present invention.

In more detail, the first epitope to be chosen would be the single epitope which occurred in the greatest number of individual isolates of the organism of interest. This single epitope would provide the greatest coverage across those isolates. When considering what to choose as a second epitope, attention should be paid to those isolates which are not yet represented by inclusion of the first epitope. In this manner, the second epitope should be chosen to provide the greatest coverage across those so far unrepresented isolates. At this point, two epitopes will have been selected. These will have been selected to provide the best coverage possible for the selection of only two epitopes. However, there may still be a group of isolates which are not yet represented. Therefore, the choice of the third epitope should address those isolates which have not yet been represented. This iterative process of choosing and selecting epitopes should be continued to achieve as high a level of coverage in terms of the number of isolates covered as possible. Preferably the process should be continued until each of the known isolates is covered.

Advantageously, if all isolates are covered yet there is still space in the composition for inclusion of further epitopes, the above process may be continued selecting further epitopes which will provide further immunological responses against the maximum number of isolates. In this way, dual responses may be generated against individual isolates. This is sometimes called the ‘double hit approach’. Clearly, the more responses which are generated against each isolate, the better chance of providing protective response in the host subject. Therefore, greater numbers of epitopes and greater numbers of hits against each isolate are preferred.

However, practical difficulties including considerations of cost in preparation of the composition dictate some limitation on the number of epitopes included in that particular composition. This limitation will vary from application to application. Furthermore, using fewer epitopes will almost always result in an easier to produce and ultimately cheaper composition. Furthermore, there may be technical advantages to reducing the number of epitopes or proteins in the compositions such as elimination of immunodominance effects, ease of balancing the responses, and/or simplification of administration. The actual limitation on the number of epitopes included in a particular composition is not important to the invention. The important principle is that when choosing the epitopes they are chosen according to the process outlined above, that is to say an iterative process in which maximising the coverage of protection is given the highest priority. In this way, whatever the actual numerical limitation on the number of epitopes included in a particular composition, a composition containing that limited number of epitopes will always provide the greatest possible coverage when the epitopes are selected according to the present invention.

Thus in one aspect the number of epitopes to be included in a particular composition will be determined before the choice of individual epitopes is made. Each individual epitope is then chosen as explained above, maximising the coverage attained with the addition of each individual epitope to the composition.

In another aspect, the coverage of a particular composition will be determined before the number of epitopes to be included in the composition is determined. Each individual epitope is then chosen as explained above, adding epitopes one at a time until the desired coverage is attained.

Naturally, many compositions may involve a compromise between a desired coverage and a preferred limitation on the number of epitopes included in the composition. The present invention advantageously enables such factors to be balanced by following the guidance given herein.

In general, the fewer antigens the composition comprises, the simpler and cheaper it will be to manufacture, administer and monitor. Therefore in some aspects a low number of epitopes will be advantageous.

In aspects of the invention when a composition is designed by coverage, then clearly a greater number of epitopes may be desirable in order to attain that coverage and the general preference for a smaller number of epitopes will be balanced to allow the desired coverage to be attained.

In another aspect, the epitopes are chosen in terms of the families of Opa's which provide best coverage. In this scenario, representative epitopes are chosen at the family level rather than at the specific epitope identity/sequence level. Thus a more general formulation scheme can be designed. Examples of this approach may be found in example 8 (table 8b) and example 9 (table 9). This approach advantageously provides for design of broader scope compositions with fewer individual components. Thus in one aspect the invention relates to a composition, preferably a vaccine composition, comprising at least one purified Opa HV1 protein epitope and at least one purified Opa HV2 protein epitope wherein each HV1 epitope in said composition is from a different HV1 family and/or wherein each HV2 epitope in said composition is from a different HV2

The composition may comprise twenty epitopes or even more. Preferably, the composition will comprise eighteen or fewer epitopes, preferably sixteen or fewer epitopes, preferably fourteen or fewer epitopes, preferably twelve or fewer epitopes, preferably eleven or fewer epitopes, preferably ten or fewer epitopes, preferably nine or fewer epitopes, preferably eight or fewer epitopes, preferably seven or fewer epitopes, preferably six or fewer epitopes, preferably five or fewer epitopes, preferably four or fewer epitopes, preferably three or fewer epitopes, preferably the composition comprises two epitopes.

The definition of an antigenically variable antigen/epitope (as compared to an antigenically conserved antigen) is well known in the art for example a variable antigen is one which differs by one or more amino acids from the sequence of the prototype antigen. As a result of this sequence difference it is likely to elicit an antibody response that is less than 100% cross-reactive between the variant and the prototype. Variable antigens will be characterised by predominance of non-synonymous over synonymous nucleotide substitutions.

### Further Components

The formulations of the present invention comprise at least one purified Opa HV1 epitope and at least one purified Opa HV2 epitope. These compositions may advantageously be supplemented with further component(s) to improve the compositions eg. to improve the efficacy of compositions such as vaccine compositions.

This third or further component may advantageously be selected from transferrin binding proteins, PorA, FetA, PorE, NspA, N. meningitidis cell surface components such as outer membrane protein(s) or other entity capable of eliciting or augmenting an immune response. Preferably the third or further component is an outer membrane protein. Preferably the third or further component is selected from the list consisting of transferrin binding proteins, PorB and NspA. Preferably the third or further component is a transferrin binding protein or PorB. Preferably the third or further component is not PorA. Preferably the third or further component is not FetA.

Preferably the compositions of the present invention do not comprise PorA.

Preferably the compositions of the present invention do not comprise FetA.

Preferably the compositions of the present invention are essentially free from both PorA and FetA.

### Compositional Formulations

The present invention provides a pharmaceutical and/or vaccine composition comprising a therapeutically effective (such as immunogenically effective) amount of the Opa HV1/HV2 epitopes of the present invention and a pharmaceutically acceptable carrier, diluent or excipient (including combinations thereof). Preferably the pharmaceutical compositions are vaccine compositions.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be administered by a number of routes.

Where the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Opa protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

### Administration

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components of the present invention may be administered alone but will generally be administered as a composition—e.g. when the components are is in admixture with a suitable pharmaceutical excipient, diluent or carrier selected with regard to the intended route of administration and standard pharmaceutical practice.

For example, the components can be administered in the form of tablets, capsules, ovules, elixirs, solutions or suspensions, which may contain flavouring or colouring agents, for immediate-, delayed-, modified-, sustained-, pulsed- or controlled-release applications.

If the administration is via a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

In a preferred aspect, the composition is delivered by injection.

It is to be understood that not all of the components of the composition need be administered by the same route. Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

The component(s) of the present invention may also be dermally or transdermally administered, for example, by the use of a skin patch.

It will be understood that these regimes include the administration of the substances sequentially, simultaneously or together.

### Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, and the individual undergoing treatment. Depending upon the need, the agent may be administered at a dose of from 0.00001 μg/Kg body weight to 5 mg/Kg body weight, preferably 0.0001 μg/Kg to 5 mg/Kg, preferably 0.001 μg/Kg to 1 mg/Kg, preferably 0.01 μg/Kg to 500 μg/Kg, preferably 0.02 μg/Kg to 300 μg/Kg body weight. Preferably the composition comprises up to about 25 μg of each Opa component.

In a preferred embodiment, a dose of approximately 3 μg is administered to a child of approximately 3-4 Kg in weight.

Preferably compositions according to the present invention are essentially free from cellular components such as polysaccharide capsule material and/or vesicles.

Preferably the vaccine compositions according to the present invention are protein vaccine compositions.

Preferably the vaccine compositions of the present invention comprise an adjuvant. Any suitable adjuvant known in the art may be employed in the present invention. The person skilled in the art will vary the adjuvant and/or quantities or proportions thereof according to the immune response required. Preferably this adjuvant is Aluminium hydrogel.

N. meningitidis

Meningococci can be classified into different genetic lineages using multilocus isolate characterisation methods and each lineage can be associated with more than one serogroup due to horizontal exchange of the siaD gene. Furthermore, whereas meningococcal populations comprise a large number of genotypes, just 7 lineages, the ST-1 complex (subgroup I), the ST-4 complex (subgroup IV), the ST-5 complex (subgroup III), the ST-8 complex (Cluster A4), the ST-11 complex (the ET-37 complex), the ST-32 complex (the ET-5 complex) and the ST-44 complex (Lineage III) have been responsible for the majority of reported meningococcal disease since the 1960s. These clonal lineages have been termed ‘hyperinvasive lineages’.

### Construction of Meningococcal Clonal-Lineage Specific Opa Vaccines/Compositions

Hyperinvasive lineage-specific compositions containing combinations of Opa proteins chosen to correspond to each hyperinvasive meningococcal lineage are constructed according to the present invention.

In outline, this is done by a method comprising

(i) the isolation of opa genes from individual isolates belonging to defined lineages of meningococci,
(ii) construction of E. coli strains expressing particular variants, and
(iii) purification and refolding of Opa proteins into their native conformations.

The Opa protein components are then assembled into combinations corresponding to the defined opa gene repertoires of the chosen individual hyperinvasive lineages.

Immune responses may vary depending on whether the protein is presented either after insertion into liposomes, or detergent micelles or in solution. Preferred is insertion into liposome vesicles. More preferred is in solution. The person skilled in the are will determine the presentation of the protein, using routine trial and error if necessary.

The presence of aluminium hydroxide and/or monophosphoryl lipid A (MPLA) adjuvants also affects immune responses. The choice of which (if any) adjuvant to use is within the abilities of the person skilled in the art. Preferred are adjuvants approved for use in children. Particularly preferred is Aluminium hydroxide.

### Monitoring of Responses

A range of immunological techniques may used to monitor responses. ELISA protocols are suitable to detect antibodies against purified, refolded recombinant Opa proteins. Preferably Opa ELISA is performed according to de Jonge M I, Vidarsson G, van Dijken H H, Hoogerhout P, van Alphen L, Dankert J, van der Ley P “Functional activity of antibodies against the recombinant OpaJ protein from Neisseria meningitidis.” Infect Immun. 2003 May; 71(5):2331-40. These can be used to determine the repertoire of antibody responses against Opa-containing compositions such as vaccines and for measurement of anti-Opa antibody titres in sera from immunised subjects. Whole cell ELISA may be used to investigate the antibody response induced following immunisation with Opa protein compared to that generated by Opa proteins in liposomes. Serum bactericidal assay may be used to measure the functional activity of anti-Opa antibodies against different clonal lineages of meningococci using exogenous complement and an opsonophagocytosis assay may be employed to demonstrate this functional characterisitic of antibody induced through immunisation.

A mouse challenge model using viability of meningococci from venous blood as the end-point of the assay may be used to evaluate/optimise compositions of the invention. Although animal models have well-recognised limitations in the evaluation of meningococcal vaccine candidates, the mouse model may be used to provide additional data to allow evaluation and/or optimisation.

The ability of anti-Opa antibodies to induce anti-adhesive immune responses may be scored. The invention may be used in the inhibiting or reducing Opa-mediated meningococcal interactions with human cells. A composition which is a vaccine composition that is both immunogenic, induces functional antibody and is cross-protective (and prevents adherence of meningococci in the nasopharynx) is particularly preferred.

### Further Advantages

The advantages of hyperinvasive lineage specific purified Opa protein compositions such as vaccine compositions of the present invention are manyfold. For example, capsule switching may occur with capsular polysaccharide-based vaccines by recombinatorial switching to an alternative antigenic (polysaccharide) type not covered by the vaccine. In the absence of a serogroup B vaccine, disease caused by meningococci that have switched to this polysaccharide type could not be controlled. The present invention addresses this problem.

Similarly, in the case of vaccines containing many variants of single proteins encoded by genes present in single copies in the meningococcal genome, escape variants generated by mutation and recombination may result in vaccine failure. By contrast, targeting of a lineage-specific multiple antigen combination such as the Opa protein repertoire provides an advantage over vaccines based on single antigens.

Although phase variation of opa gene loci may result in changes to the expression state of each locus, lineage specific Opa vaccines advantageously include the proteins commonly expressed from all opa loci and therefore advantageously eliminate the effects of phase variation.

Antibodies directed to Opa epitopes on meningococci have been found to be bactericidal, showing protective immunity. Additionally, antibodies directed against Opa may reduce or block adherence of meningococci in the nasopharynx since this protein is important in meningococcal adhesion.

Indeed, the Opa proteins are a primary means of intimate attachment between the meningococcus and its host via their interaction with the N-terminal domain of the human carcinoembryonic antigen cell adhesion molecule (CEACAM) protein family. This interaction is central to carriage of meningococci in the human nasopharynx and plays a key role in invasion through the mucosa—a step in the pathogenetic sequence that precedes meningococcal disease.

Compositions of the present invention advantageously elicit immune reactions which interfere with adherence to host receptors.

The invention is now described by way of examples which are intended to illustrate the invention and are not meant to be limiting, in which reference is made to the following figures:

FIG. 1 shows a diagram of Opa protein.

Furthermore, the appended sequence listing shows sequence data for opa alleles used herein. The invention also relates to these opa alleles per se.

### EXAMPLE 1

### Population Structure of Opa Proteins

This example describes a novel approach to development of a serogroup B meningococcal vaccine using data from meningococcal population biology to develop a vaccine targeted specifically at hyperinvasive lineages of meningococci using combinations of Opa proteins.

Opa proteins exhibit genetic and antigenic diversity. In order to investigate whether this diversity possessed structure and to explore the evolutionary processes involved in its generation, a systematic survey of the diversity in the Opa proteins was performed using the population structure of the meningococcus as a theoretical framework. The meningococcal Opa proteins are encoded by 3-4 phase variable opa gene loci per isolate (the opa gene repertoire) and individual isolates have the potential to express these proteins from all four loci. Whereas the level of diversity in the opa genes was found to be high and localised in three variable regions corresponding to 3 of the putative surface exposed loops, it was also non-randomly structured at a number of levels. In the 7 major hyperinvasive lineages, using isolates chosen to represent the global, temporal and genetic diversity in each lineage, there was a stable association of opa gene repertoire, with particular alleles consistently present at each locus in individual clonal lineages (data from the ST-32 complex is included as an example in Table A.). This structure was maintained during periods of global spread spanning up to three decades and was also found in the opa gene repertoires of isolates taken from a collection of carriage strains, many of which belonged to genotypes which have never been associated with cases of meningococcal disease.

Table A illustrates stable association of opa alleles in the hyperinvasive ST-32 (ET-5) complex of meningococci. A recombinant purified protein composition such as a vaccine composition corresponding to the Opa repertoire of the lineage in this example contains each of the opa alleles defined in this table.

In a preferred example of the present invention such a composition would contain the proteins encoded by alleles opa96, opa185, opa288, opa147, opa335 and opa218.

Allele opa335 is highly similar to opa 185, differing only in the SV region. Thus in another preferred example of this composition opa335 is omitted from the composition (whilst opa185 is retained).

The isolate Z6418 represents a defined subclone of this lineage. Consequently, all other isolates would contain at least three proteins covered by the composition.

TABLE A

| Isolate | Lineage | Year | Country | opaA | opaB | opaD | opaJ |
|---|---|---|---|---|---|---|---|
| Z3842 | ST-32 complex | 1976 | Norway | 96 | 288 | 147 | 218 |
| Z4695 | ST-32 complex | 1981 | Norway | 96 | 288 | 71 | 218 |
| Z4696 | ST-32 complex | 1982 | Norway | 96 | 313 | 147 | 218 |
| Z4664 | ST-32 complex | 1984 | Holland | 96 | 335 | 147 | 218 |
| Z4672 | ST-32 complex | 1985 | Holland | 96 | 185 | 147 | 218 |
| Z4680 | ST-32 complex | 1985 | DDR | 96 | 185 | 147 | 218 |
| Z4699 | ST-32 complex | 1985 | Norway | 96 | 185 | 185 | 218 |
| Z4239 | ST-32 complex | 1987 | Norway | 96 | 185 | 147 | 218 |
| Z4706 | ST-32 complex | 1987 | Chile | 96 | 185 | 147 | 218 |
| Z6418 | ST-32 complex | 1992 | Cuba | 261 | 185 | 147 | 322 |

### Methods:

Meningococcal isolate collections and amplification of opa genes: The hyperinvasive ST-8, ST-11, ST-32 and ST-44 complexes have all been previously associated with the serogroup B capsular polysaccharide and isolates from these are chosen for use in this investigation. All isolates are taken from existing collections and have all been characterised previously by multilocus sequence typing (MLST). The opa gene repertoires for these have also been determined. Locus specific PCR amplification is used to amplify each opa gene for insertion into a suitable expression vector.

Preparation of Opa Proteins: Methods for Cloning, Expression, Purification and refolding of Opa proteins are essentially as described in Prince S M, Feron C, Janssens D, Lobet Y, Achtman M, Kusecek B, Bullough P A and Derrick J P (2001 Acta Crystallogr D Biol Crystallogr. August; 57(Pt 8):1164-6 “Expression, refolding and crystallization of the OpcA invasin from Neisseria meningitidis.”.

The expression vectors are constructed and purification of expressed proteins is undertaken.

The 4 Opa proteins from ST-32 (ET-5 complex) are purified and refolded and it is anticipated that less than 16 proteins will provide coverage for the 4 lineages.

Preparation of Compositions and Immunisation: Lineage Specific Combinations of purified, refolded recombinant Opa proteins are prepared for immunisation in a variety of formulations. These include purified refolded recombinant protein, and proteins incorporated into liposomes. All preparations may be used both with and without addition of the adjuvant aluminium hydroxide.

Mice are immunised with single and multiple clonal lineage-specific purified Opa compositions to determine the most efficient preparation for protection against single and multiple hyperinvasive meningococcal lineages. Immune responses to each of the above preparations are tested in BALB/c female mice at 7 weeks of age. Blood samples are taken before primary immunisation by tail-vein bleed. Preliminary dose-ranging experiments are performed to determine optimal dose: 1, 5, 10 and 20 μg will be evaluated. We then immunise 4 groups of 12 mice with each of the above preparations (one preparation per group). Individual mice receive 3 doses of protein in each preparation at 14 days apart starting at day 0. We compare the results to groups of 12 control mice, immunised accordingly with either the same amount of liposome vesicle or adjuvant alone. Mice are terminally bled 14 days after the final immunisation, sera are prepared and stored at −80° C. in 100 μl aliquots.

Characterisation of anti-Opa antibody: anti-Opa serum antibody responses are characterised by Opa protein ELISA, whole cell ELISA, serum bactericidal assay and a flow cytometric opsonophagocytosis assay using established protocols such as those published by S. Romero-Steiner, such as the Streptococcus pneumoniae opsonophagocytosis using differentiated HL-60 cells (Promyelocytic Leukemia Cell Line) Laboratory Protocol prepared by: S. Steiner, Ph.D.; D. LiButti, B. S.; H. L. Keyserling, M.D.; G. M. Carlone, Ph.D. of the Centers for Disease Control and Prevention, and Emory University Atlanta, Ga., preferably the ‘draft 2.0 version’ is used which can be found at http://www.vaccine.uab.edu/refer/cdcops3.htm. Clearly in the context of the present invention Neisseria meningitidis is used rather than Streptococcus pneumonia. Furthermore, in a preferred embodiment NB4 cells are used in place of HL-60 cells.

Mouse challenge model: Once an immunogenic Opa combination composition is constructed and evaluated using the immunogenicity studies described above, 6-8 week old mice are immunised with three doses of the candidate vaccine composition 14 days apart and challenged with 10⁷ colony forming units of N. meningitidis from hyperinvasive lineages intraperitoneally 2 weeks later. The “protective” activity of immunisation is assessed by comparing viability of meningococci from tail vein bleeds at different time points using established protocols such as those described by Gorringe et al in ‘Methods in Molecular Medicine—Meningococcal disease’ edited by Andrew Pollard and Martin Maiden, Humana Press Inc., 2001: Chapter 17 “Animal models for meningococcal disease.” by Andrew R. Gorringe, Karen M. Reddin, Pierre Voet and fan T. Poolman. This technique is preferably modified by taking bleeds and determining the bacterial counts per ml blood to monitor the course of the infection.

### EXAMPLE 1a

### Purified Opa Vaccine Against the ST-32 Clonal Complex of Neisseria meningitidis

An Opa vaccine against the ST-32 clonal complex, based on analysis of the Opa repertoire of 10 isolates belonging to this lineage contains 4 Opa proteins (Table 1). A vaccine of this formulation would include at least 1 epitope at each locus in 8/10 isolates: including 8 epitopes across all 4 opa loci in 7/10 isolates (70% of all epitopes), 6 epitopes across 4 loci in one isolate (Z6418, in which the HV1 variants of the opaA and opaJ loci would not be recognised) and 6 epitopes across 3 loci in 2 isolates (Z4695, in which the opaD locus would not be recognised and isolate Z4696 in which the opaB locus would not be recognised).

TABLE 1
Formulation for a purified recombinant Opa-combination
vaccine against the ST-32 complex

| opa allele | SV | HV1 | HV2 | HVRC |
|---|---|---|---|---|
| 147 | 2-2 | 1A-2 | 8-1 | 3 |
| 185 | 5-1 | 10-5 | 3-1 | 37 |
| 96 | 2-1 | 19-10 | 14-3 | 75 |
| 218 | 2-1 | 19-10 | 14-5 | 75 |

### EXAMPLE 2

### Purified Opa Vaccine Against the ST-11 Clonal Complex of Neisseria meningitidis

An Opa vaccine against the ST-11 clonal complex, based on analysis of the Opa repertoire of 10 isolates belonging to this lineage, contains 6 Opa proteins (Table 2). A vaccine of this formulation would include at least one epitope at each locus in 8/10 isolates: 8 epitopes across all 4 opa loci in 4/10 isolates, 7 epitopes across 4 loci in 4 isolates (the HV1 epitope of the opaB locus in isolates Z4242, Z4243 and the HV1 epitope at the opaJ locus of isolates Z4323 and Z4631 would not be recognised), 6 epitopes across 3 loci in one isolate (the opaB locus in isolate Z6417 would not be recognised) and 4 epitopes across 2 loci in the remaining isolate (the opaB and opaJ loci in isolate Z4765 would not be recognised).

Analysis of 53 isolates belonging to the closely related ET-15 clone of the ST-11 complex (collected from the Czech Republic in 1993) revealed a difference in the epitopes at the opaA locus. Allele opa83 (present in 50/51 isolates for which an opaA allele could be determined) encoded the SV: 3, HV1:5, HV2:18 which was not present in the collection of 69 isolates. The opaB allele was opa11, encoding the same HV epitopes as opa34 (Table 2) but differing in the SV region, encoding SV 3-1. This allele was present in 45/49 isolates for which an opaB allele could be determined (the opaB locus in 3 isolates was disrupted by the presence of an insertion element and so the opaB allele could not be determined). At the opaD locus, the opa132 allele was present in 50/50 isolates for which an opaD sequence could be determined. The sequence at the opaJ locus could not be determined for any of the ST-11 complex isolates in this collection.

TABLE 2
Formulation for a purified recombinant Opa-combination
vaccine against the ST-11 complex

| opa allele | SV | HV1 | HV2 | HVRC |
|---|---|---|---|---|
| 244 | 4-3 | 5-5 | 8C-1 | 24 |
| 132 | 2-2 | 11-2 | 1-6 | 40 |
| 34 | 4-3 | 18-3 | 14-1 | 69 |
| 317 | 2-2 | 19-1 | 14-1 | 75 |
| 279 | 2-2 | 1A-5 | 9B-2 | 4 |
| 83 | 3-4 | 5-2 | 18-1 | 27 |

### EXAMPLE 3

### Purified Opa Vaccine Against the ST-5 Clonal Complex of Neisseria meningitidis

An Opa vaccine against the ST-5 clonal complex, based on analysis of the Opa repertoire of 11 isolates belonging to this lineage, would contain 4 Opa proteins (Table 3). A vaccine of this formulation would include at least 2 epitopes at each locus in 9/11 isolates: 8 epitopes across all 4 opa loci in 9/11 isolates and 6 epitopes across 3 loci in the remaining 2 isolates (the opa281 allele (SV2-1, HV1:6-1, HV2:9-2, HVRC29) at the opaB locus of isolates Z3524 and Z3771 would not be recognised).

TABLE 3
Formulation for a purified recombinant Opa-combination
vaccine against the ST-5 complex

| opa allele | SV | HV1 | HV2 | HVRC |
|---|---|---|---|---|
| 253 | 2-10 | 1-3 | 8A-5 | 3 |
| 242 | 2-1 | 2-3 | 1-8 | 8 |
| 127 | 4-3 | 3-6 | 1-1 | 11 |
| 296 | 2-1 | 17-3 | 17-8 | 67 |

### EXAMPLE 4

### Purified Opa Vaccine Against the ST-4 Clonal Complex of Neisseria meningitidis

An Opa vaccine against the ST-4 clonal complex, based on analysis of the Opa repertoire of 10 isolates belonging to this lineage, would contain 4 Opa proteins (Table 4). This clonal complex has 3 opa loci, as the gene at the opaJ locus is deleted, and so the total number of HV-region epitopes in isolates belonging to this lineage is 6 (3 loci, 2 epitopes per locus). A vaccine of this formulation would include at least 1 epitope at all loci in 10/11 isolates: all 6 epitopes across all 3 opa loci in 10/11 isolates and 6 epitopes across 3 loci in the remaining isolate, Z1001 (a member of the ST-4 complex by MLST but which belongs to the serogroup A, subgroup IV-2 clone, rather than subgroup IV-1, by MLEE) in which the opaB locus would not be recognised.

TABLE 4
Formulation for a purified recombinant Opa-combination
vaccine against the ST-4 complex

| opa allele | SV | HV1 | HV2 | HVRC |
|---|---|---|---|---|
| 242 | 2-1 | 2-3 | 1-8 | 8 |
| 253 | 2-10 | 1-3 | 8A-5 | 3 |
| 297 | 2-2 | 11-2 | 9B-1 | 42 |
| 296 | 2-1 | 17-3 | 17-8 | 67 |

### EXAMPLE 5

### Purified Opa Vaccine Against the ST-44 Clonal Complex of Neisseria meningitidis

An Opa vaccine against the ST-44 clonal complex, based on analysis of the Opa repertoire of 9 isolates belonging to this lineage, would contain 5 Opa proteins (Table 5). A vaccine of this formulation would include at least 1 epitope at all opa loci 6/9 isolates: 8 epitopes across all 4 opa loci in 4/9 isolates, 7 epitopes across 4 loci in 2 isolates (the HV1 epitope of the opaD locus in isolate Z6420 and of the opaB locus in isolate Z6422 would not be recognised), 6 epitopes across 3 loci in 3 isolates (the opaA locus in isolate Z6421 and the opaD locus in the isolates Z6423 and Z6427 would not be recognised).

TABLE 5
Formulation for a purified recombinant Opa-combination
vaccine against the ST-44 complex

| opa allele | SV | HV1 | HV2 | HVRC |
|---|---|---|---|---|
| 264 | 2-1 | 1B-4 | 6-1 | 2 |
| 245 | 2-1 | 1-5 | 9A-2 | 4 |
| 213 | 2-1 | 3-6 | 1-1 | 11 |
| 201 | 2-2 | 4-1 | 7-3 | 20 |
| 265 | 3-2 | 7-1 | 5-9 | 31 |

### EXAMPLE 6

### Purified Opa Vaccine Against the ST-1 Clonal Complex of Neisseria meningitidis

An Opa vaccine against the ST-1 clonal complex, based on analysis of the Opa repertoire of 10 isolates belonging to this lineage, would contain 13 Opa proteins (Table 6). A vaccine of this formulation, without the 11 other Opa, would include at least 1 epitope at all loci in 5/10 isolates: 8 epitopes across all 4 opa loci in 5/10 isolates, 6 epitopes across 3 loci in 1 isolate (the opaA locus of isolate Z1099 would not be recognised), 5 epitopes across 3 loci in 3 isolates (the opaB locus and the HV2 epitope at the opaJ locus in isolate Z1275, the opaB locus and the HV1 epitope at the opaA locus of isolate Z1466, the opaJ locus and the HV2 epitope of the opaB locus of isolate Z5010 would not be recognised). In the remaining isolate, Z1092, only the 2 epitopes at the opaJ locus would be recognised. For coverage of at least one epitope at all loci in all isolates in this lineage would require a further 7 Opa (alleles opa250, opa96, opa304, opa340, opa298, opa311, opa345).

TABLE 6
Formulation for a purified recombinant Opa-combination
vaccine against the ST-1 complex

| opa allele | SV | HV1 | HV2 | HVRC |
|---|---|---|---|---|
| 77 | 2-2 | 1B-4 | 6-1 | 2 |
| 243 | 2-1 | 1-1 | 9-3 | 4 |
| 325 | 2-1 | 11-1 | 1-4 | 40 |
| 252 | 4-2 | 13-1 | 17-7 | 54 |
| 145 | 4-2 | 12-2 | 21-1 | 50 |
| 326 | 2-2 | 18-7 | 14-1 | 69 |

Plus 7 other Opa

### EXAMPLE 7

### Purified Opa Vaccine Against the ST-8 Clonal Complex of Neisseria meningitidis

An Opa vaccine against the ST-8 clonal complex, based on analysis of the Opa repertoire of 8 isolates belonging to this lineage, would contain 5 Opa proteins (Table 7). A vaccine of this formulation would include at least 1 epitope at all loci in 7/8 isolates: 8 epitopes across all 4 opa loci in 6/8 isolates, 7 epitopes across 4 loci in one isolate (the HV2 epitope at the opaB locus of isolate Z4671 would not be recognised) and 6 epitopes across 3 loci in the remaining isolate (the opaB locus of isolate Z6411 would not be recognised).

TABLE 7
Formulation for a purified recombinant Opa-combination
vaccine against the ST-8 complex

| opa allele | SV | HV1 | HV2 | HVRC |
|---|---|---|---|---|
| 292 | 2 | 5 | 18 | 27 |
| 161 | 2 | 11 | 1 | 40 |
| 260 | 2 | 3 | 6 | 12 |
| 258 | 4 | 19 | 14 | 75 |
| 246 | 2 | 15 | 12 | 58 |

### EXAMPLE 8a

### Purified Opa Vaccine Against the Hyperinvasive Clonal Complexes of Neisseria meningitidis Commonly Found the UK (HV1, HV2 Variants)

An Opa vaccine against the ST-8, ST-11, ST-32 and ST-44 clonal complexes, based on analysis of the Opa repertoire of a total of 37 isolates belonging to these lineages would contain over 20 Opa (including the most prevalent 18 Opa in Table 8a).

TABLE 8a
Formulation for a purified recombinant
Opa-combination UK vaccine

| opa allele | SV | HV1 | HV2 | HVRC |
|---|---|---|---|---|
| 34 | 4-3 | 18-3 | 14-1 | 69 |
| 83 | 3-4 | 5-2 | 18-1 | 27 |
| 96 | 2-1 | 19-10 | 14-3 | 75 |
| 132 | 2-2 | 11-2 | 1-6 | 40 |
| 147 | 2-2 | 1A-2 | 8-1 | 3 |
| 161 | 2-2 | 11-1 | 1-4 | 40 |
| 185 | 5-1 | 10-5 | 3-1 | 37 |
| 201 | 2-2 | 4-1 | 7-3 | 20 |
| 213 | 2-1 | 3-6 | 1-1 | 11 |
| 244 | 4-3 | 5-5 | 8C-1 | 24 |
| 245 | 2-1 | 1-5 | 9A-2 | 4 |
| 246 | 2-2 | 15-3 | 12-2 | 58 |
| 258 | 4-3 | 19-4 | 14-2 | 75 |
| 260 | 2-2 | 3-2 | 6-2 | 12 |
| 264 | 2-1 | 1B-4 | 6-1 | 2 |
| 265 | 3-2 | 7-1 | 5-9 | 31 |
| 279 | 2-2 | 1A-5 | 9B-2 | 4 |
| 317 | 2-2 | 19-1 | 14-1 | 75 |

### EXAMPLE 8b

### Purified Opa Vaccine Against the Hyperinvasive Clonal Complexes of Neisseria meningitidis Commonly Found the UK (HV1, HV2 Families)

An Opa vaccine against the ST-8, ST-11, ST-32 and ST-44 clonal complexes, based on analysis of the Opa repertoire of a total of 37 isolates belonging to these lineages would contain 14 Opa (Table 8b) when the formulation is based on HV (epitope) families rather than on HV1 and HV2 variants. A vaccine of this formulation would include at least 1 epitope at all loci in 36/37 isolates: 8 epitopes across all 4 opa loci in 31/37 isolates, 7 epitopes across 4 opa loci in 5 isolates (the HV1 epitope of the opaD locus in isolate Z4765; the HV2 epitope of the opaB locus of isolate Z4671; the HV2 epitope of the opaD locus of isolate Z4695 and the HV2 epitope of the opaB locus in isolates Z6411 and Z6417 would not be recognised). In the remaining isolate, Z6421, the opaA locus would not be recognised.

TABLE 8b
Formulation for a purified recombinant
Opa-combination UK vaccine

| opa allele | SV family | HV1 family | HV2 family | HVRC |
|---|---|---|---|---|
| 34 | 4 | 18 | 14 | 69 |
| 83 | 3 | 5 | 18 | 27 |
| 147 | 2 | 1A | 8 | 3 |
| 161 | 2 | 11 | 1 | 40 |
| 185 | 5 | 10 | 3 | 37 |
| 201 | 2 | 4 | 7 | 20 |
| 213 | 2 | 3 | 1 | 11 |
| 244 | 4 | 5 | 8C | 24 |
| 245 | 2 | 1 | 9A | 4 |
| 246 | 2 | 15 | 12 | 58 |
| 258 | 4 | 19 | 14 | 75 |
| 264 | 2 | 1B | 6 | 2 |
| 265 | 3 | 7 | 5 | 31 |
| 279 | 2 | 1A | 9B | 4 |

### EXAMPLE 9

### Purified Opa Vaccine Against the Hyperinvasive Clonal Complexes of Neisseria meningitidis Commonly Found in Africa (HV1, HV2 Families)

An Opa vaccine against the ST-1, ST-4, ST-5 and ST-11 clonal complexes, based on analysis of the Opa repertoire of a total of 42 isolates belonging to these lineages, contains the 11 Opa proteins (Table 9). A vaccine of this formulation would recognise at least 1 epitope at all loci in 39/42 isolates. The only epitopes not recognised would be the opaD locus of isolates Z1275, Z5010 and the opaB locus of Z417.

TABLE 9
Formulation for a purified recombinant
Opa-combination African vaccine

| opa allele | SV family | HV1 family | HV2 family | HVRC |
|---|---|---|---|---|
| 77 | 2 | 1B | 6 | 2 |
| 253 | 2 | 1 | 8A | 3 |
| 243 | 2 | 1 | 9 | 4 |
| 242 | 2 | 2 | 1 | 8 |
| 127 | 4 | 3 | 1 | 11 |
| 244 | 4 | 5 | 8C | 24 |
| 83 | 3 | 5 | 18 | 27 |
| 297 | 2 | 11 | 9B | 42 |
| 296 | 2 | 17 | 17 | 67 |
| 34 | 4 | 18 | 14 | 69 |
| 317 | 2 | 19 | 14 | 75 |

### EXAMPLE 10

### Demonstration of Immunogenicity and Bactericidal Effects

As will be apparent from the above description, the present invention relates to compositions comprising purified Opa protein epitopes. In particular, the invention relates to pharmaceutical compositions comprising same, preferably immunogenic pharmaceutical compositions comprising same, and most preferably to vaccine compositions comprising same.

In this example, the induction of immune responses using compositions of the invention is demonstrated. Thus the immunogenicity of the compositions is demonstrated. Furthermore, according to the most preferred embodiments, the induction of bactericidal immune responses is demonstrated ie. the effectiveness of the vaccine compositions of the invention is shown.

We have been able to demonstrate bactericidal activity. Bactericidal activity is the serological correlate of protection. Thus, bactericidal activity induced using the compositions of the invention illustrates the protective effects produced by same (ie. effective vaccination).

Referring to Example 2, a total of six Opa proteins were chosen to represent the ST-11 complex of Neisseria meningitidis. These are presented in Table 2.

In this example, we demonstrate the effectiveness of using such protein compositions to induce immune response and in vaccinating to induce protection against N. meningitidis. In this example, the first protein to be used in pre-clinical trials to investigate safety and immunogenicity in mice is OpaB from the ST-11 complex isolate 38VI.

In this example, the OpaB protein from isolate 38VI was encoded by opa allele 34. Full characterisation details of this allele are as follows: opa34: SV:4-3, HV1 :18-3, HV2:14-1.

Groups of 6-8 week old, female BalbC mice are immunised in three doses given three weeks apart with either (i) Freund's adjuvant (Freund's complete adjuvant for the first dose and Freund's incomplete adjuvant for the second and third doses). (ii) 2.5 μg OpaB_{38VI} in Freund's adjuvant (given as in (i)).

All mice survived until the end of the experiment at which time serum was collected from each mouse by terminal, tail vein bleed. All serum, including a portion of serum (50 μl) from each mouse which was pooled, was stored below 70° C.

Immunogenicity was investigated by ELISA (enzyme linked immunosorbent assay) and SBA (serum bactericidal antibody) assay. Antibody against OpaB_{38VI} was detected in serum from mice immunised with this protein in ELISA, whereas no antibody responses were detected in control sera.

For SBA experiments, aliquots of pooled serum samples were thawed, heated at 56° C. for 30 minutes to deactivate complement and diluted 1 in 4 with HBSS. These samples were then serially diluted between 1 in 16 to I in 16384 (final reaction concentrations) before incubation with 1000 colony forming units (cfu) of isolate 38VI in the presence of baby rabbit complement (final concentration in reaction: 1 in 20 dilution from stock) for 1 hour at 37° C., 5% CO₂.

In initial SBA experiments, bactericidal activity against isolate 38VI was not observed at a dilution of 1 in 16. The activity is thus tested at higher concentrations.

Dot blot experiments indicated that expression of this Opa protein variant, or of potentially cross reacting variants, may be higher on other isolates from a set of eleven epidemiologically diverse ST-11 complex meningococci. Serum bactericidal activity against three other ST-11 complex isolates in this collection (D1, M597 and 0037/93) was tested. Under these reaction conditions, a serum bactericidal titre of >1 in 256 was obtained against isolates 0037/93 and M597. Bactericidal activity was also observed against isolate D1.

Immunisation experiments using OpaA_{38VI} and OpaD_{38VI} are conducted as above for OpaB. Serum samples are collected and analysed in the same manner.

Immunisation with the remaining three ST-11 proteins: OpaB_{NG P20}, OpaA_{0046/93} and OpaJ_{38VI} is conducted. Demonstration of safety and analysis of immunogenicity against the representative collection of ST-11 complex meningococci is carried out.

These experiments show that immunisation with a single Opa protein from ST11 complex induced bactericidal antibody in sera that had activity against multiple ST11 complex isolates. Thus, both immunogenicity and protective effect are demonstrated for compositions according to the present invention.

Combinations of Opa proteins such as those disclosed herein with particular reference to Example 2 (table 2) provide even broader protection.

All publications mentioned in the above specification are herein incorporated by reference. Various modifications and variations of the described methods and system of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A pharmaceutical composition comprising at least one purified Opa HVI protein epitope and at least one purified Opa HV2 protein epitope.

2. The pharmaceutical composition according to claim 1 further comprising at least one purified Opa SV protein epitope.

3. A pharmaceutical composition according to claim 1 wherein said epitopes are selected from the epitopes defined in Table A and Tables 1-9.

4. A pharmaceutical composition according to claim 3 wherein said epitopes are selected from the epitopes defined in a single table selected from Table A and Tables 1-9.

5. A pharmaceutical composition according to claim 4 wherein said composition comprises each of the epitopes defined in said single table.

6. A pharmaceutical composition according to claim 1 wherein the composition comprises epitopes encoded by Opa alleles presented in the sequence listing.

7. A pharmaceutical composition according to claim 1 wherein the composition comprises epitopes encoded by the Opa alleles defined in a single table selected from Table A and Tables 1-9.

8. A pharmaceutical composition according to claim 7 wherein said composition comprises each of the epitopes defined in said single table.

9. A pharmaceutical composition according to claim 1 further comprising one or more components selected from the group consisting of transferrin binding proteins, PorB and NspA.

10. A pharmaceutical composition according to claim 9 wherein said further component is selected from the group consisting of transferring binding protein and PorB.

11. A pharmaceutical composition according to claim 10 wherein said further component is PorB.

12. A composition according to claim 1 which is a vaccine composition.

13. A vaccine composition comprising OpaB protein encoded by opa allele 34.

14. A composition comprising a combination of Opa epitopes as set out in a single table selected from Table A and Tables 1-9.

15. A composition according to claim 1 comprising a combination of Opa epitopes.

16. A composition according to claim 14 which is a vaccine composition.

17. A composition according to claim 16 wherein said composition is an outer membrane vesicle vaccine.

18. A composition according to claim 1 comprising at least one purified protein selected from the group consisting of OpaA, OpaB, OpaD and OpaJ.

19. A composition according to claim 18 comprising at least two purified proteins selected from the group consisting of OpaA, OpaB, OpaD and OpaJ.

20. A composition according to claim 19 comprising at least three purified proteins selected from the group consisting of OpaA, OpaB, OpaD and OpaJ.

21. A composition according to claim 20 comprising purified OpaA, OpaB, OpaD and OpaJ protein.

22. A method of immunising a subject against Neisseria meningitidis infection comprising administering to said subject an effective amount of a composition according to claim 1.

23. A method of inducing an immune response against Neisseria meningitidis in a subject comprising administering to said subject an effective amount of a composition according to claim 1.

24. An isolated nucleic acid comprising nucleotide sequence encoding a N. meningitidis Opa protein, said nucleotide sequence being selected from the Opa allele sequences SEQ ID NO.: 1-37.
